(19) United States
(12) Patent Application Publication
(10) Pub. No.: US 2023/0039395 A1
(43) Pub. Date: Feb. 9, 2023

(54) LSD AND PSILOCYBIN DOSE EQUIVALENCE DETERMINATION

(71) Applicant: Universitätsspital Basel, Basel (CH)

(72) Inventors: Matthias Emanuel LIECHTI, Oberwil (CH); Friederike Sophie HOLZE, Naters (CH)

(73) Assignee: Universitätsspital Basel, Basel (CH)

(21) Appl. No.: 17/734,601

(22) Filed: May 2, 2022

Related U.S. Application Data
(60) Provisional application No. 63/228,870, filed on Aug. 3, 2021.

(51) Int. Cl. A61K 49/00; A61K 31/48; A61K 31/675

(52) U.S. Cl. CPC A61K 49/0004; A61K 31/48; A61K 31/675

(57) ABSTRACT

A method of treating a patient with a psychedelic, by administering either a dose of LSD to the patient that is equivalent to a known dose of psilocybin with desired acute and therapeutic effects, or administering a dose of psilocybin to the patient that is equivalent to a known dose of LSD with desired acute and therapeutic effects. A method of treating a patient with LSD, by administering a dose of LSD to the patient equivalent to those of psilocybin known to be associated with positive long-term therapeutic outcomes. A method of determining a dose of a psychedelic or the dose-equivalence to another psychedelic to be administered to an individual, by administering a dose of a psychedelic to an individual, determining positive acute effects and negative acute effects in the individual, and adjusting the dose to provide more positive acute effects than negative acute effects in the individual.

### GRANT INFORMATION

Research in this application was supported by a grant of the Swiss National Science Foundation (32003B_185111/1) to Matthias Liechti.

### BACKGROUND OF THE INVENTION

### 1. Technical Field

The present invention relates to compositions and methods for identifying dose ranges for lysergic acid diethylamide (LSD) and psilocybin to produce specific subjective drug effects in treating medical conditions.

### 2. Background Art

LSD and psilocybin are hallucinogens or psychedelics capable of inducing exceptional subjective effects such as a dream-like alteration of consciousness, pronounced affective changes, enhanced introspective abilities, visual imagery, pseudo-hallucinations, synesthesia, mystical-type experiences, and experiences of ego dissolution (Holze et al., 2021; Liechti, 2017; Passie et al., 2008). Psychedelics including LSD and psilocybin have also newly been termed psychoplastogens because these substances exhibit neuroregenerative effects that can contribute to their therapeutic effects (Ly et al., 2018).

LSD has been widely used for recreational and personal purposes (Krebs & Johansen, 2013). Additionally, LSD is increasingly used in experimental research (Carhart-Harris et al., 2016c; Dolder et al., 2016; Liechti, 2017; Preller et al., 2017; Schmid et al., 2015) and for the treatment of psychiatric patients (Gasser et al., 2014; Gasser et al., 2015). However, correct dosing of LSD to induce specific responses is a problem. At present, there are no completed modern clinical studies in patients describing the effects of well-defined doses of LSD. The only modern completed study using LSD in patients (Gasser et al., 2014; Gasser et al., 2015) used a formulation of LSD which was later shown to be unstable and thus the true dose used in not known (Holze et al., 2019; Liechti & Holze, 2021).

The formulations of LSD mostly used in recent clinical research did not have long-term stability and the doses actually administered to humans were therefore likely lower or at least unclear in these studies (Barrett et al., 2018; Dolder et al., 2016; Kraehenmann et al., 2017a; Kraehenmann et al., 2017b; Preller et al., 2018; Preller et al., 2017; Preller et al., 2019; Schmid et al., 2015). Specifically longer-term stability data beyond the full study duration were unavailable for the capsules that were used in several previous studies (Dolder et al., 2016; Dolder et al., 2017; Kraehenmann et al., 2017b; Liechti et al., 2017; Mueller et al., 2018; Mueller et al., 2017a; Mueller et al., 2017b; Preller et al., 2017; Schmid et al., 2015; Schmid & Liechti, 2018; Schmidt et al., 2017). Further, after administration of the 200 μg dose in the form of two 100 μg capsules, iso-LSD was detected in plasma (Steuer et al., 2017), indicating that this inactive decomposition product of LSD was possibly already present in the capsules at the time of their use (although possible formation in the plasma samples cannot be completely excluded). The plasma AUC24 values of LSD and iso-LSD of 21 and 9.2 ng×h/ml (Steuer et al., 2017) indicate that on average 30% of the LSD may have isomerized to inactive iso-LSD in the capsules. Thus, the actual administered doses of LSD may have been 70 and 140 μg LSD base rather than the indicated 100 and 200 μg, respectively. The AUC_{∞} values in the previous studies that used 100 and 200 μg doses were 61% and 76%, respectively, of the values that were expected based on confirmed 96 μg LSD doses used in a later study (Holze et al., 2019) and assuming similar bioavailability. Finally, analytical tests of four unused old LSD capsules that were performed years after study completion suggested a marked reduction of LSD content (remaining amount of LSD=22±7 μg), indicating a lack of longer-term stability of LSD in this form and that the actual LSD doses that were used were likely already lower than indicated during the studies. Notably, a decrease in content by 15% or even 25% in single capsules would still be compatible with content uniformity, which was documented during production of the capsules. In summary, based on the results of different quality-control measures, analytical findings (including pharmacokinetic data (Holze et al., 2019)), and the clinical effects of the different formulations (Dolder et al., 2017), it is likely that previous studies actually used approximately 60-70 (not 100) μg and 140-150 (not 200) μg of LSD base, corresponding to approximately 80 and 175 μg of LSD tartrate.

Another consideration is that doses of LSD that were reported in previous studies may not have been very precise or may not have reflected the actual exposure of LSD in the body. This is notable in recent studies that used intravenous dosing with 75 μg hydrophobic LSD base in saline because objective measures of exposure to LSD (i.e., plasma concentrations) were lacking, and the bioavailability of the solution is unknown (Carhart-Harris et al., 2016b; Carhart-Harris et al., 2015; Carhart-Harris et al., 2016c; Kaelen et al., 2015; Tagliazucchi et al., 2016). The clinical response to 75 μg of intravenous LSD was not significantly different from the oral 100 μg dose that was used in previous studies (Carhart-Harris et al., 2016b; Liechti, 2017; Liechti et al., 2017), indirectly indicating similar exposure that is comparable to an oral dose of 60-70 μg LSD base.

In the light of the above noted data, even scientifically published data on LSD doses previously used is not sufficiently correct to guide safe and efficacious dose selection for medical treatment.

The acute effects of well-defined doses of LSD have been described only in healthy subjects and in a few studies (Holze et al., 2019; Holze et al., 2021; Holze et al., 2020) but not in most previous studies and not in any studies in patients. In contrast, there are many published modern studies using psilocybin at defined doses in psychiatric patients including patients with major depression (Carhart-Harris et al., 2016a; Davis et al., 2021; Griffiths et al., 2016; Roseman et al., 2017; Ross et al., 2016), anxiety disorder or anxiety associated with terminal illness (Griffiths et al., 2016; Grob et al., 2011; Ross et al., 2016), and in different forms of addiction (Bogenschutz, 2013; Bogenschutz et al., 2015; Garcia-Romeu et al., 2019; Garcia-Romeu et al., 2015; Johnson et al., 2014; Johnson et al., 2016).

There is no valid data on the equivalent doses of LSD and psilocybin. Thus, it is unclear how much LSD would be needed to produce similar effects of psilocybin acutely and longer-term. Comparative doses of LSD and psilocybin are not known for healthy subjects nor patients.

It has been shown that the acute effects of psychedelics are comparable in healthy subjects and patients (Schmid et al., 2021). Thus, acute effects in patients can be assumed to be similar to effects in healthy subjects and known effects in healthy subjects can critically inform on the dosing in patients.

Additionally, positive acute subjective psychedelic experiences after administration of psilocybin are correlated with its long-term therapeutic benefits in patients with depression or addiction (Garcia-Romeu et al., 2015; Griffiths et al., 2016; Roseman et al., 2017). This means that the acute effects of a serotonergic psychedelic in humans can be used to predict, at least in part, the therapeutic outcome in patients. Even in healthy subjects, positive acute responses to psychedelics have been shown to be linked to more positive long-term effects on well-being (Griffiths et al., 2008; Schmid & Liechti, 2018).

Acute effects that may contribute to positive long-term effects of psychedelics including psilocybin and LSD are effects that are thought to enhance the therapeutic relationship including increased openness, trust, feelings of connectedness or emulsion with persons, insight in psychological problems and stimulation of neuroregenerative processes as described in detail elsewhere (Vollenweider & Preller, 2020). Importantly, higher Oceanic Boundlessness and lower anxiety ratings that were acutely induced by psilocybin were previously shown to predict better treatment efficacy in patients with depression, anxiety, and tobacco dependence (Garcia-Romeu et al., 2015; Griffiths et al., 2016; Roseman et al., 2017; Ross et al., 2016).

There remains a need for defining the doses of psilocybin and LSD that produce mostly positive acute effects predictive of therapeutic benefits and to the define the doses of LSD that correspond to the doses of psilocybin shown to be therapeutically beneficial in patients.

There is no modern valid information on whether effects of different serotonergic hallucinogens are similar or different regarding the quality of the subjective effects. For example, LSD and psilocybin both bind to serotonin (5-HT)_{2A} receptors (Glennon et al., 1992; Rickli et al., 2016) and these 5-HT_{2A} receptors are thought to primarily mediate their hallucinogenic effects (Barrett et al., 2017; Holze et al., 2021; Kraehenmann et al., 2017a; Kraehenmann et al., 2017b; Preller et al., 2016; Preller et al., 2017; Vollenweider et al., 1998). However, there are also differences in the pharmacological profiles between LSD and psilocybin. Psilocin inhibits the 5-HT transporter (SERT) whereas LSD stimulates the D₁₋₃ receptors (Rickli et al., 2016). Whether these molecular differences have an influence on the subjective effects and on alterations of consciousness has not yet been studied in humans. Both substances are used in psychiatric research to induce alterations of mind and there are now several modern studies using either LSD or psilocybin. However, differences in their acute clinical effects have not been studied and in particular the two substances have not been compared face to face using modern and validated psychometric measures and study methodology (Liechti, 2017). Therefore, there remains a need for a comparison of the effects of LSD and psilocybin

### SUMMARY OF THE INVENTION

The present invention provides for a method of treating a patient with a psychedelic, by administering either a dose of LSD to the patient that is equivalent to a known dose of psilocybin with desired acute and therapeutic effects or administering a dose of psilocybin to the patient that is equivalent to a known dose of LSD with desired acute and therapeutic effects and treating the patient.

The present invention provides for a method of treating a patient with LSD, by administering a dose of LSD to the patient equivalent to those of psilocybin known to be associated with positive long-term therapeutic outcomes.

The present invention provides for a method of determining a dose of a psychedelic or the dose-equivalence to another psychedelic to be administered to an individual, by administering a dose of a psychedelic to an individual, determining positive acute effects and negative acute effects in the individual, adjusting the dose to provide more positive acute effects than negative acute effects in the individual, and equating the dose to an equivalent dose of a second psychedelic.

### DESCRIPTION OF THE DRAWINGS

Other advantages of the present invention are readily appreciated as the same becomes better understood by reference to the following detailed description when considered in connection with the accompanying drawings wherein:

FIG. 1A is a drawing of the chemical structure of LSD and FIG. 1B is a drawing of the chemical structure of psilocybin;

FIG. 2 is a schematic of the study design and participant flow;

FIG. 3A is a graph showing acute any drug effect and FIG. 3B is a graph showing good drug effects of LSD and psilocybin;

FIG. 4A is a graph showing acute drug high and FIG. 4B is a graph showing feeling stimulated induced by LSD and psilocybin;

FIG. 5A is a graph showing drug liking and FIG. 5B is a graph showing feeling happy induced by LSD and psilocybin;

FIG. 6A is a graph showing subjective bad drug effect and FIG. 6B is a graph showing fear induced by LSD and psilocybin;

FIG. 7A is a graph showing feeling content and FIG. 7B is a graph showing trust induced by LSD and psilocybin;

FIG. 8A is a graph showing feeling talkative and FIG. 8B is a graph showing changes in perception of time (sense of time) induced by LSD and psilocybin;

FIG. 9A is a graph showing feeling open and FIG. 9B is a graph showing subjective concentration induced by LSD and psilocybin;

FIG. 10A is a graph showing subjective speed of thinking and FIG. 10B is a graph showing feeling close to others induced by LSD and psilocybin pretreatment;

FIG. 11A is a graph showing wanting to be hugged and FIG. 11B is a graph showing wanting to hug someone induced by LSD and psilocybin;

FIG. 12A is a graph showing desire to be alone and FIG. 12B is a graph showing desire to be with others induced by LSD and psilocybin;

FIG. 13 is a graph showing ego dissolution induced by LSD and psilocybin;

FIG. 14A is a graph showing performance-oriented activity, FIG. 14B is a graph showing inactivity, and FIG. 14C is a graph showing well-being induced by LSD and psilocybin;

FIG. 15A is a graph showing extraversion, FIG. 15B is a graph showing introversion, and FIG. 15C is a graph showing emotional excitation induced by LSD and psilocybin;

FIG. 16 is a graph showing anxiety induced by LSD and psilocybin;

FIGS. 17A-17C are graphs showing effects of LSD and psilocybin on the 5-Dimensions of Altered States Scale main scales and subscales, FIG. 17A is a graph showing oceanic boundlessness, FIG. 17B is a graph showing anxious ego dissolution, and FIG. 17C is a graph showing visionary restructuralization;

FIG. 18A is a graph showing effects of LSD and psilocybin on the Mystical Experience Questionnaire (MEQ43), and FIG. 18B is a graph showing effects of LSD and psilocybin on MEQ30;

FIG. 19A is a graph showing effects of LSD and psilocybin on systolic blood pressure and FIG. 19B is a graph showing diastolic blood pressure;

FIG. 20A is a graph showing effects of LSD and psilocybin on heart rate and FIG. 20B is a graph showing effects of LSD and psilocybin on body temperature;

FIG. 21 is a graph showing effects of LSD and psilocybin on rate pressure product;

FIG. 22A is a graph showing effects of LSD and psilocybin pupil size, FIG. 22B is a graph showing effects on pupil size after light, and FIG. 22C is a graph showing effects on pupil constriction;

FIG. 23A is a graph representing the effects of LSD and psilocybin on cortisol plasma concentrations and FIG. 23B is a graph representing the effects of LSD and psilocybin on brain prolactin plasma concentrations;

FIG. 24 is a table listing the most frequent complaints during the acute effects of LSD and psilocybin;

FIG. 25 is a table of the treatment identification (unblinding) of the study

FIG. 26 is a table of the mean values and statistics of the subjective effects of LSD and psilocybin on the visual analog scales (VAS);

FIG. 27 is a table of the mean values and statistics of the subjective effects of LSD and psilocybin on the Adjective Mood Rating Scale (AMRS);

FIG. 28 is a table of the mean values and statistics of the acute mind-altering effects of LSD and psilocybin in the 5 Dimensions of Altered States of Consciousness Scale (5D-ASC);

FIG. 29 is a table of the mean values and statistics of the of the acute effects of LSD and psilocybin in the Mystical Experience Questionnaire (MEQ43 and MEQ30);

FIG. 30 is a table of the mean values and statistics of the acute autonomic, adverse, and endocrine effects of LSD and psilocybin; and

FIG. 31 is a table of dose equivalence of psilocybin and LSD.

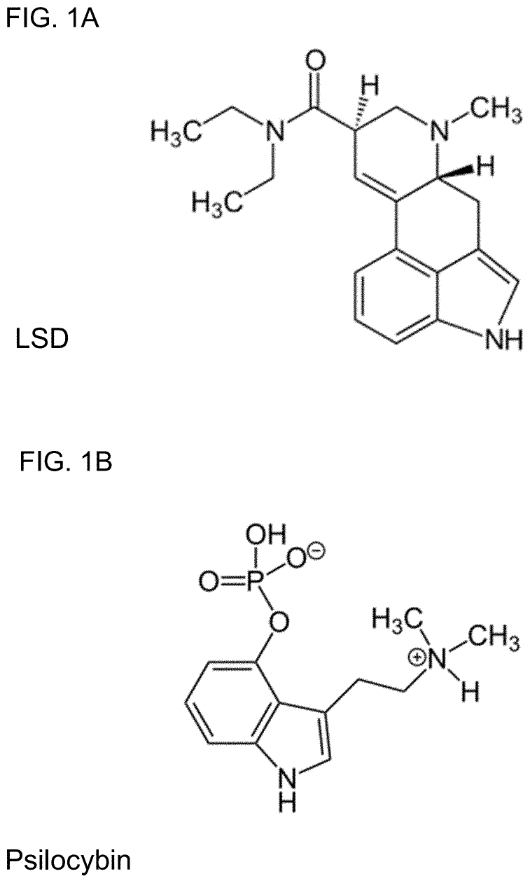

### DETAILED DESCRIPTION OF THE INVENTION

The present invention relates to the use of specific doses of LSD and psilocybin to define the dose equivalence to induce comparable acute and longer-term therapeutic effects using these substances as medical treatments. Defining the dose equivalence of LSD and psilocybin is important to compare study efficacy data between the two substances and deriving for example a dose of LSD to be used in a study or a patient based on the equivalence to a dose of psilocybin show to be effective in a study. The dose equivalence defined in the present invention relates to equivalence in a group of subjects or patients such as in a study population rather than to individual doses. Thus, the benefits are greatest when using the present equivalence results to estimate acute beneficial and adverse effects for study populations. While effects of LSD and psilocybin may vary in single individuals their dose equivalence is expected to vary less. Thus, if a dose of LSD has pronounced effects in one subject the equivalent dose of psilocybin is expected to also have pronounced effects and vice versa. The equivalence of the two substances was determined within the present invention in Example study 1 and can now be applied for future studies.

The induction of an overall positive acute response to the psychedelic is critical because several studies showed that a more positive experience is predictive of a greater therapeutic long-term effect of the psychedelic (Garcia-Romeu et al., 2015; Griffiths et al., 2016; Ross et al., 2016). Even in healthy subjects, positive acute responses to psychedelics including LSD have been shown to be linked to more positive long-term effects on well-being (Griffiths et al., 2008; Schmid & Liechti, 2018).

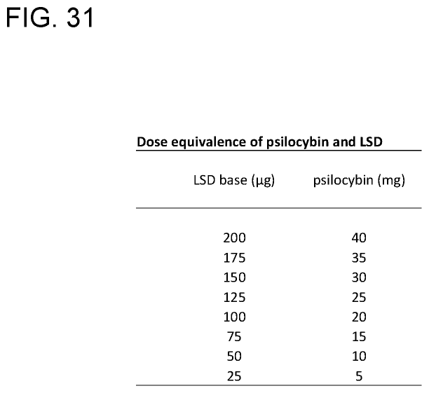

Generally, the present invention provides for a method of dosing and treating patients with a psychedelic, by administering LSD or psilocybin at a specific dose and the method allows for determining therapeutically equivalent doses of the two psychedelics. The overall goal of the present invention is to improve the positive over negative acute subjective effect response to a psychedelic and to define the doses of LSD and psilocybin that produce comparable positive and negative acute subjective effect to improve dosing of the two substances. More specifically, the present invention provides for a method of treating a patient with a psychedelic, by administering either a dose of LSD to the patient that is equivalent to a known dose of psilocybin with desired acute and therapeutic effects or administering a dose of psilocybin to the patient that is equivalent to a known dose of LSD with desired acute and therapeutic effects. FIG. 31 lists equivalent doses of LSD and psilocybin. Most preferably, the patient is being treated for depression, anxiety, or addiction or any other disorder where data for one of the substances has been generated or will be generated and an equivalent dose of the other has to be defined.

Because more therapeutic data is available on psilocybin compared with LSD, the invention also allows for defining the doses of LSD equivalent to that of psilocybin having the desired therapeutic effects in a given patient population.

“Positive acute effects” as used herein refers primarily to an increase in subjective rating of “good drug effect” and can also include ratings of “drug liking”, “well-being”, “oceanic boundlessness”, “experience of unity”, “spiritual experience”, “blissful state”, “insightfulness”, any “mystical-type experience” and positively experienced “psychedelic effects”, and “aspects of ego-dissolution” if experienced without anxiety. Preferably, the dose administered maximizes the positive acute effects in the patient.

“Negative acute effects” as used herein refers primarily to subjective ratings of “bad drug effect” and “anxiety” and “fear” and may additionally include increased ratings of “anxious ego-dissolution”, or descriptions of acute paranoia or states of panic an anxiety as observed by others. Preferably, the dose administered minimizes the negative acute effects in the patient.

Main Findings

The compounds of the present invention are administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compounds of the present invention can be administered in various ways. It should be noted that they can be administered as the compound and can be administered alone or as an active ingredient in combination with pharmaceutically acceptable carriers, diluents, adjuvants and vehicles. The compounds can be administered orally, transcutaneously, subcutaneously or parenterally including intravenous, intramuscular, and intranasal administration. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses or a continuous dose over a period of several hours.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The present invention also provides for a method of determining a dose of a psychedelic or the dose-equivalence to another psychedelic to be administered to an individual, by administering a dose of a psychedelic, determining positive acute effects and negative acute effects in the individual, adjusting the dose to provide more positive acute effects than negative acute effects in the individual, and equating the dose to an equivalent dose of a second psychedelic. The individual can be healthy and the method can be used to predict doses for unhealthy individuals. This method can be used to determine long term dosing and dose schedules. The psychedelics used in determining a dose of a second psychedelic can be, but are not limited to, LSD, psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), ibogaine, ketamine, salts thereof, tartrates thereof, solvates thereof, isomers thereof, analogs thereof, homologues thereof, or deuterated forms thereof.

In addition, dose-finding for clinical trials is difficult and time and money consuming. It would be much easier and cost-effective and rapid if a method were available to define the dose and dose equivalence to be used in patients already in Phase 1 studies in healthy subjects. Evaluating the acute effects in healthy subjects with a focus on positive acute over negative effects as a documented predictor of long-term outcome in patients can greatly facilitate the dose-finding for future Phase 2 and Phase 3 studies in patient populations. Therefore, this method can be used in predicting and determining doses and/or dose equivalence for psychedelics for clinical trials.

Psychedelics can induce neuroregeneration (Ly et al., 2018). Plasma brain-derived neurotrophic factor (BDNF) levels are a possible biomarker for neurogenesis (Haile et al., 2014). Higher BDNF levels were associated with lower depression ratings after administration of ayahuasca (de Almeida et al., 2019). The present invention provides for a method of defining equivalent effects of psychedelics on neuroregeneration by using a dose that produces the same effects on circulating BDNF and comparable subjective effects assuming that these are predictive biomarkers for regenerative and therapeutic effects.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass any and all variations which become evident as a result of the teaching provided herein. The data described herein and in Example 1 has been published after submission of the provisional patent (Holze et al. 2022).

### EXAMPLE 1

The following key findings were observed. Effects of LSD and psilocybin were overall very similar with the exception that the duration of effects of psilocybin was shorter compared with LSD.

LSD produced comparable overall effects and positive drug effects at the 100 and 200 μg doses. However, the 200 μg produced more bad drug effects, ego dissolution, and anxiety than the 100 μg dose of LSD.

The dose of 30 mg of psilocybin was similar in terms of effect intensity to both the 100 or 200 μg doses of LSD. Effects of 30 mg psilocybin were typically nominally between those of the 100 and 200 μg LSD doses and not significantly different from either dose. The only exception, where effects of psilocybin 30 mg were different from LSD was ineffability on the MEQ which was greater after 200 μg LSD compared with 30 mg psilocybin.

Additionally, psilocybin (30 mg) increased blood pressure more than LSD and LSD (200 μg) increased heart rate more than psilocybin. The rate-pressure product was similarly increased by LSD and psilocybin, indicating comparable overall cardiovascular stimulant properties. LSD and psilocybin increased cortisol and PRL plasma concentrations in line with their serotonergic profile.

The Example study and findings are described in more detail below.

Materials and Methods

Study design: The study used a double-blind, placebo-controlled, cross-over design with five experimental test sessions to investigate the responses to 1) placebo 2) 100 μg LSD, 3) 200 μg LSD, 4) 15 mg psilocybin, and 5) 30 mg psilocybin. The washout periods between sessions were at least 10 days. The study was registered at ClinicalTrials.gov (NCT03604744).

Participants: Twenty-eight healthy subjects (14 men and 14 women; mean age±SD: 34±9 years; range: 25-52 years). Participants who were younger than 25 years old were excluded from participating in the study. Additional exclusion criteria were age >65 years, pregnancy (urine pregnancy test at screening and before each test session), personal or family (first-degree relative) history of major psychiatric disorders (assessed by the Semi-structured Clinical Interview for Diagnostic and Statistical Manual of Mental Disorders, 4th edition, Axis I disorders by a trained psychiatrist), the use of medications that can interfere with the study medications (e.g. antidepressants, antipsychotics, sedatives), chronic or acute physical illness (abnormal physical exam, electrocardiogram, or hematological and chemical blood analyses), tobacco smoking (>10 cigarettes/day), lifetime prevalence of illicit drug use >10 times (except for Δ⁹-tetrahydrocannabinol), illicit drug use within the last 2 months, and illicit drug use during the study (determined by urine drug tests).

Study Drugs: LSD (D-lysergic acid diethylamide base, high-performance liquid chromatography purity >99%; Lipomed AG, Arlesheim, Switzerland) was administered as oral solution in units containing 100 μg LSD in 1 mL of 96% ethanol (Holze et al., 2019). The analytically confirmed LSD base content per vial was 84.46 μg±0.98, n=10 compliant with the target dose and uniformity requirements. Psilocybin was prepared as capsules containing 5 mg of analytically pure (HPLC purity 99%) psilocybin dihydrate (ReseaChem GmbH, Burgdorf, Switzerland) and mannitol filler. Formulations plus matching placebos were prepared by a GMP facility (Apotheke Dr. Hysek, Biel, Switzerland) according to GMP guidelines. The analytically confirmed psilocybin content per capsule was 4.61 mg±0.09, n=10 compliant with the target dose and uniformity requirements. Blinding to treatment was guaranteed by using a double-dummy method, with identical capsules and vials that were filled with mannitol and ethanol, respectively, as placebo. At the end of each session and at the end of the study, the participants were asked to retrospectively guess their treatment assignment.

Study procedures: The study included a screening, five 25 hour test sessions, and an end-of-study visit. The sessions were conducted in a calm standard hospital room. Only one research subject and one investigator were present during the test sessions. The test sessions began at 8:00 AM. The subjects then underwent baseline measurements. LSD or psilocybin or placebo was administered at 9:00 AM. The outcome measures were repeatedly assessed for 24 hours. A standardized lunch and dinner were served at 1:30 PM and 6:00 PM, respectively. The subjects were under constant supervision by an investigator until 9:00 PM. Thus, the subjects were never alone during the first 12 hours after drug administration, and the investigator was in a room next to the subject for up to 24 hours. The subjects were sent home the next day at 9:15 AM.

Subjective drug effects: Subjective effects were assessed repeatedly using visual analog scales (VASs) 1 hour before and 0, 0.25, 0.5, 0.75, 1, 1.5, 2, 2.5, 3, 4, 5, 6, 7, 8, 9, 10, 11, 12, 14, 16, and 24 hours after drug administration. The VASs included “any drug effect,” “good drug effect,” “bad drug effect,” “drug liking,” “drug high,” “stimulated,” “fear,” “content”, “happy”, “trust”, “talkative”, “open”, “concentration”, “speed of thinking”, “sense of time”, “closeness”, “want to be hugged”, “want to hug”, “want to be alone”, “want to be with others”, and “ego dissolution,” (Holze et al., 2021). The VASs were presented as 100-mm horizontal lines (0-100%), marked from “not at all” on the left to “extremely” on the right. The VASs for “content”, “happy”, “trust”, “talkative”, “open”, “concentration”, “speed of thinking”, “sense of time”, “closeness”, “want to be hugged”, “want to hug”, “want to be alone”, and “want to be with others” were bidirectional (±50%). Marked from “not at all” on the left (−50), to “normal” in the middle (0), to “extremely” on the right (+50) and “slowed” (−50) and “racing” (+50) for “perception of time”. The 5D-ASC scale (Dittrich, 1998; Studerus et al., 2010) was administered 24 hours after LSD administration to retrospectively rate alterations in waking consciousness induced by the drugs. Mystical experiences were assessed using the German version (Liechti et al., 2017) of the 100-item States of Consciousness Questionnaire (SOCQ) (Griffiths et al., 2006) that includes the 43-item and newer 30-item MEQ (MEQ43 (Griffiths et al., 2006) and MEQ30 (Barrett et al., 2015)). The 60-item Adjective Mood Rating Scale (AMRS) (Janke & Debus, 1978) was administered 1 hour before and 3, 6, 9, 12, and 24 hours after drug administration.

Autonomic and adverse effects: Blood pressure, heart rate, and tympanic body temperature were repeatedly measured 1 hour before and 0, 0.25, 0.5, 0.75, 1, 1.5, 2.5, 3, 4, 5, 6, 7, 8, 9, 10, 11, 12, 14, 16, and 24 hours after drug administration as previously described in detail (Hysek et al., 2010). Adverse effects were systematically assessed 1 hour before, 12 and 24 hours after drug administration using the 66-item List of Complaints (Zerssen, 1976). This scale yields a total adverse effects score and reliably measures physical and general discomfort.

Endocrine effects: Plasma concentrations of cortisol, prolactin (PRL), oxytocin, and brain-derived neurotrophic factor (BDNF) were determined as described elsewhere (Holze et al., 2021; Schmid et al., 2015). Cortisol and PRL were measured before and 2.5 hours after drug administration. BDNF was measured before and three times after drug administration.

Plasma drug concentrations: Blood was collected into lithium heparin tubes 1 hour before and 0, 0.25, 0.5, 0.75, 1, 2, 3, 4, 6, 8, 10, 12, 14, 16, and 24 hours after LSD administration. The blood samples were immediately centrifuged, and the plasma was subsequently stored at −80° C. until analysis. Plasma concentrations of LSD, O-H-LSD, psilocin (active metabolite of the prodrug psilocybin), and 4-hydroxy-indolic acetic acid (4-HIAA) were determined using a validated ultra-high-performance liquid chromatography tandem mass spectrometry method as described previously in detail (Holze et al., 2019; Kolaczynska et al., 2021).

Pharmacokinetic analyses and pharmacokinetic-pharmacodynamic modeling: Pharmacokinetic parameters were estimated using non-parametric methods in Phoenix WinNonlin 6.4 (Certara, Princeton, N.J., USA) as described previously in detail (Holze et al., 2019).

Data analysis: Peak (Eₘₐₓ and/or Eₘᵢₙ) or peak change from baseline (ΔEₘₐₓ) values were determined for repeated measures. The values were then analyzed using repeated-measures analysis of variance (ANOVA), with drug as within-subjects factor, followed by Tukey post hoc comparisons using Statistica 12 software (StatSoft, Tulsa, Okla., USA). The criterion for significance was p<0.05.

Results

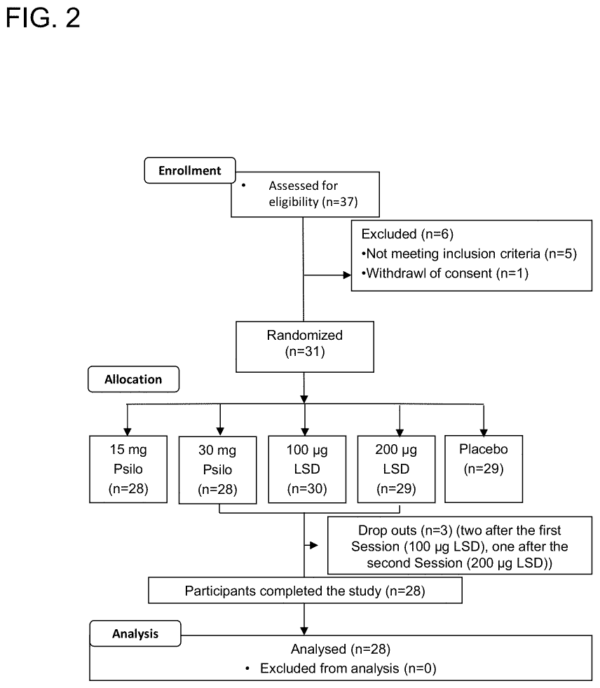

The study overview and participant flow are shown in FIG. 2.

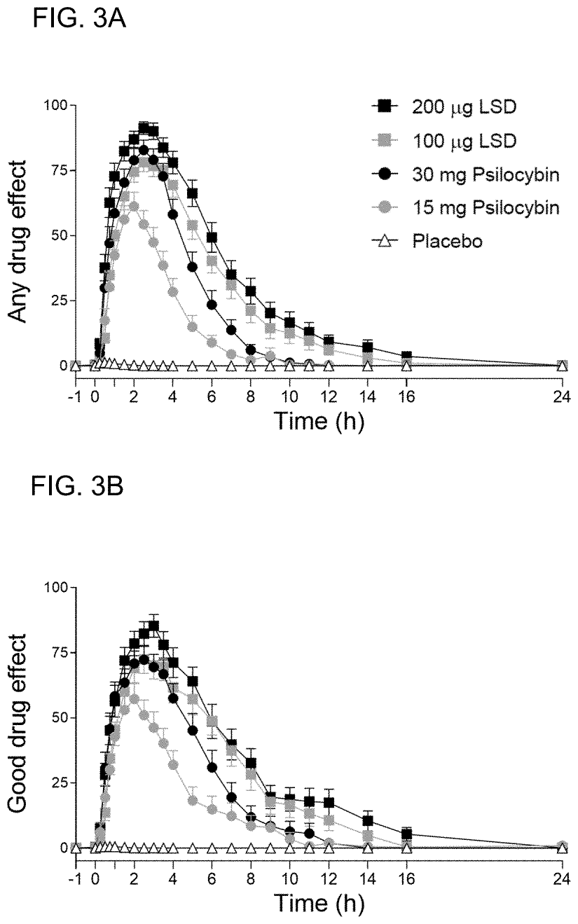

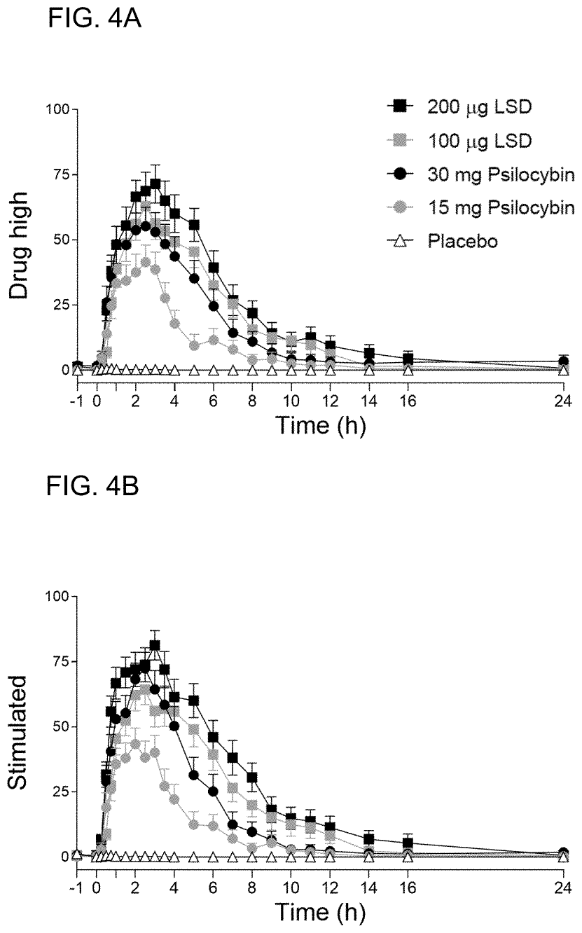

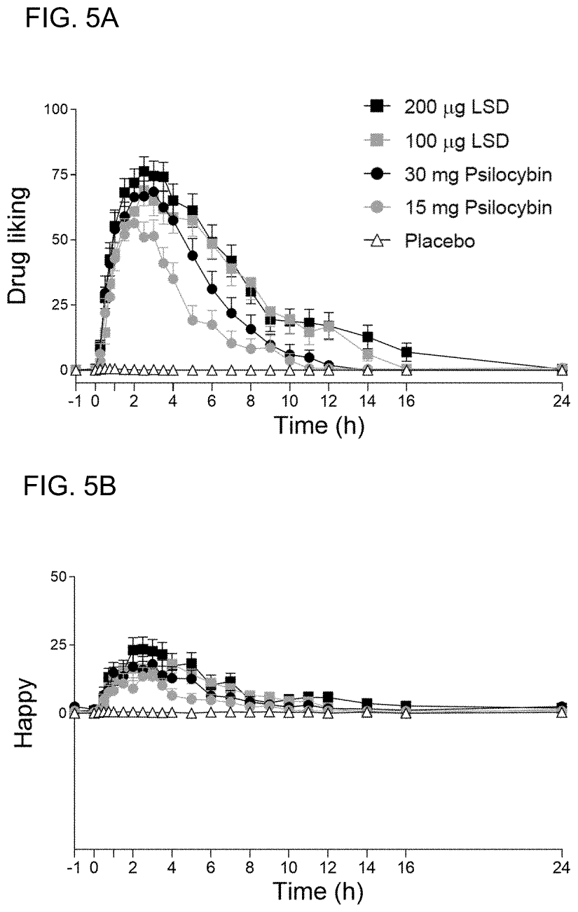

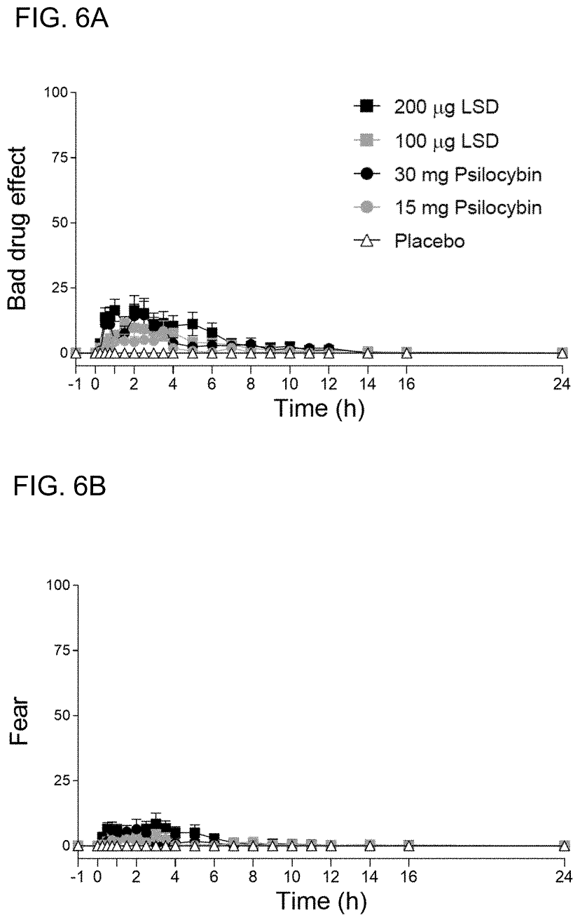

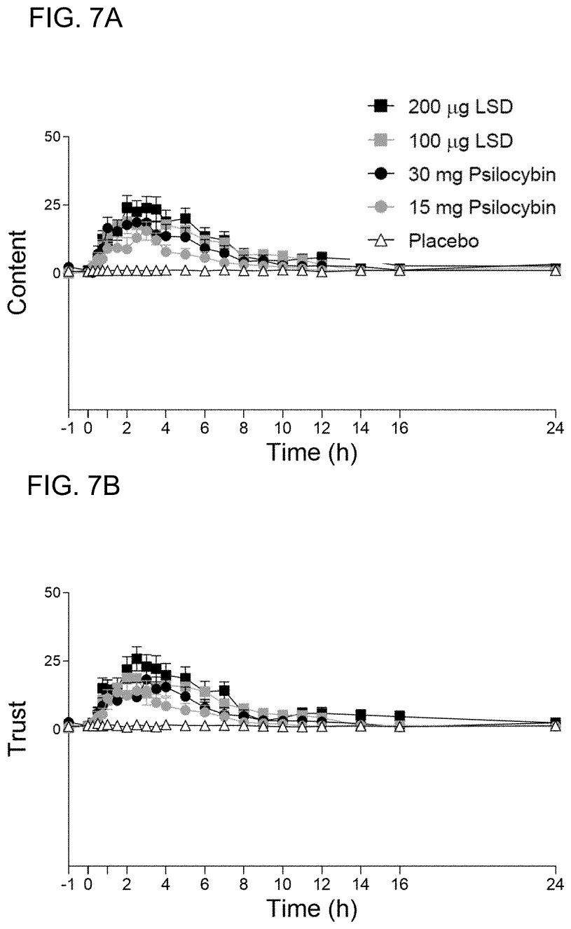

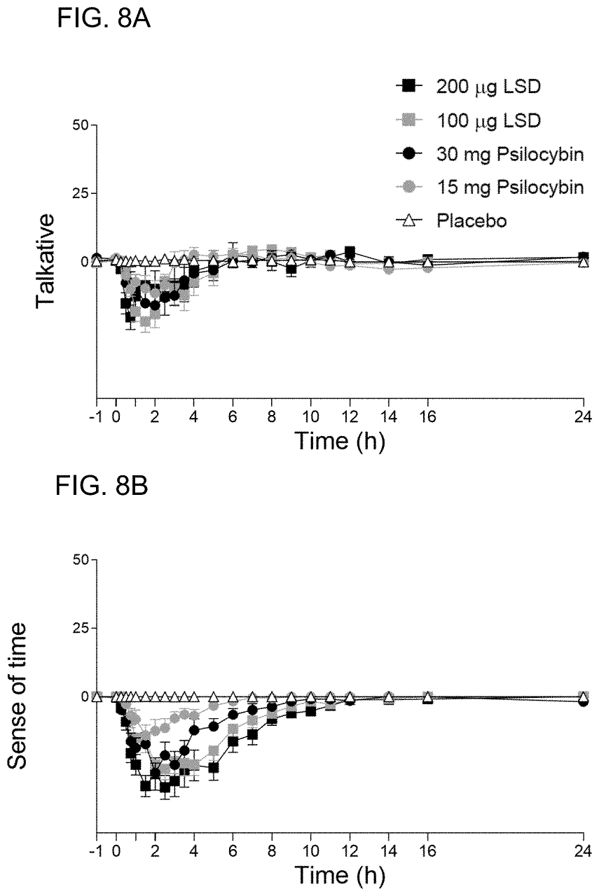

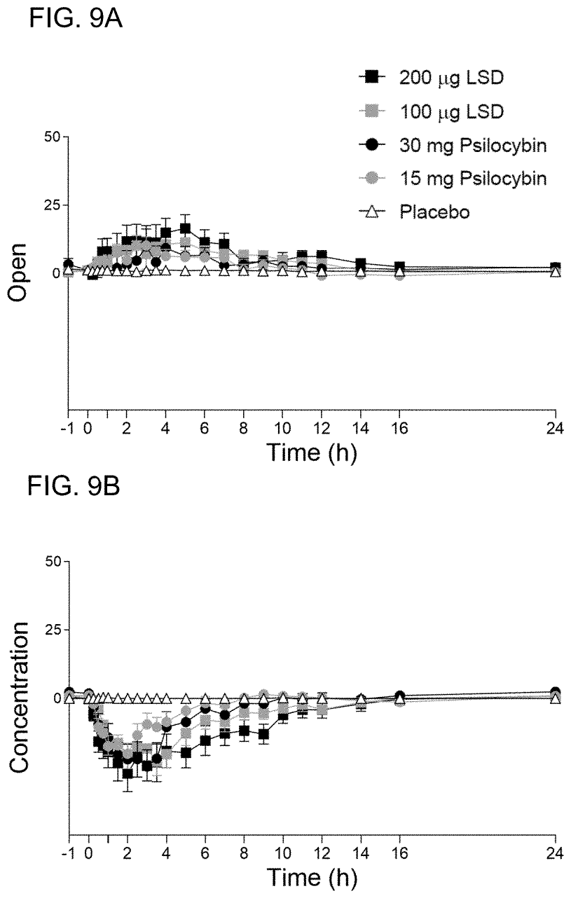

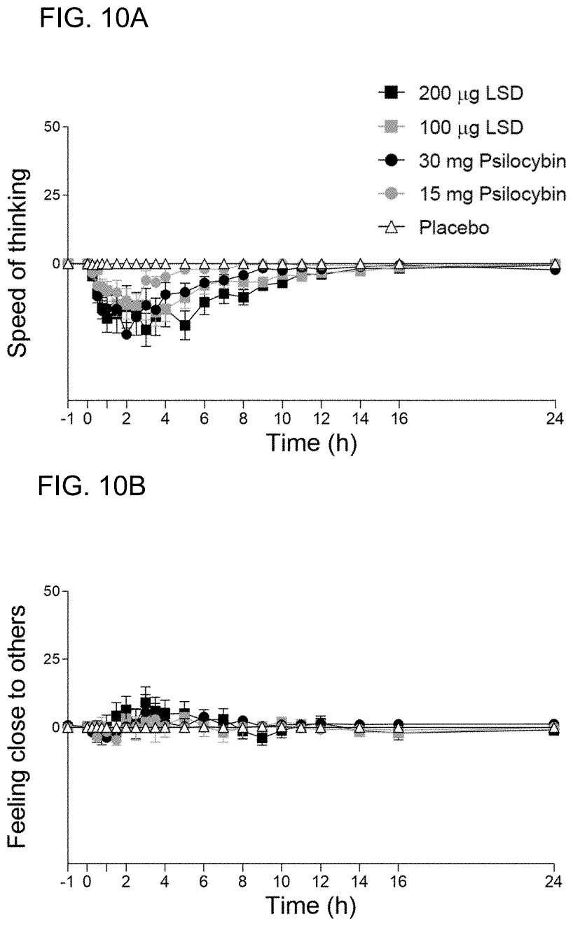

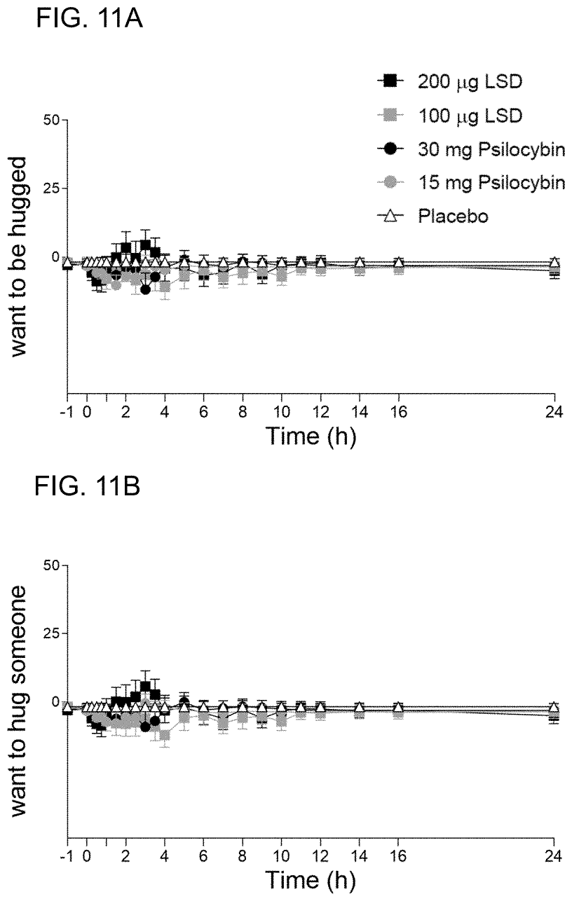

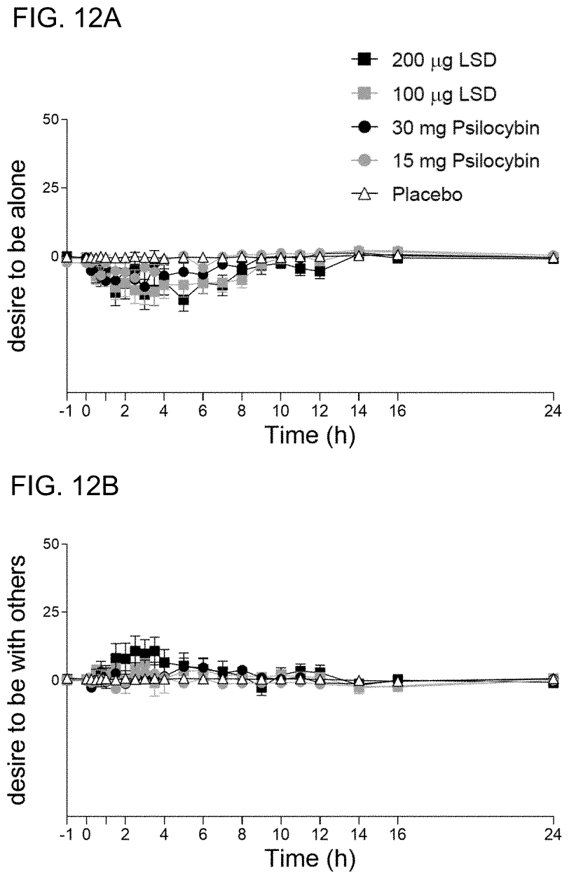

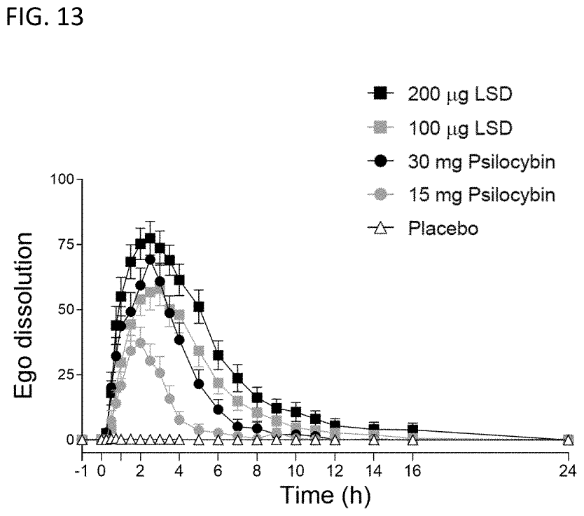

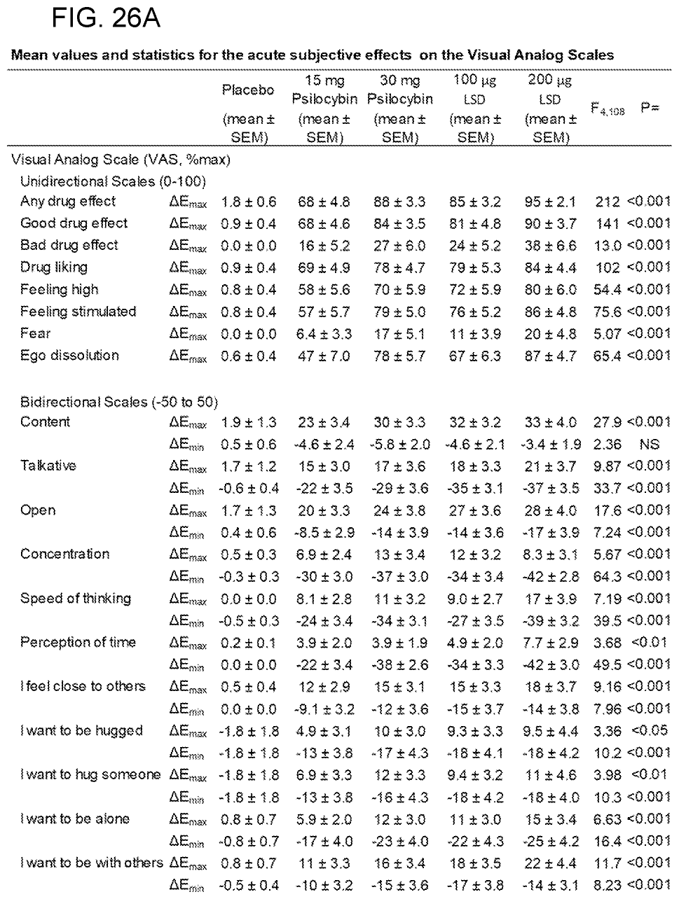

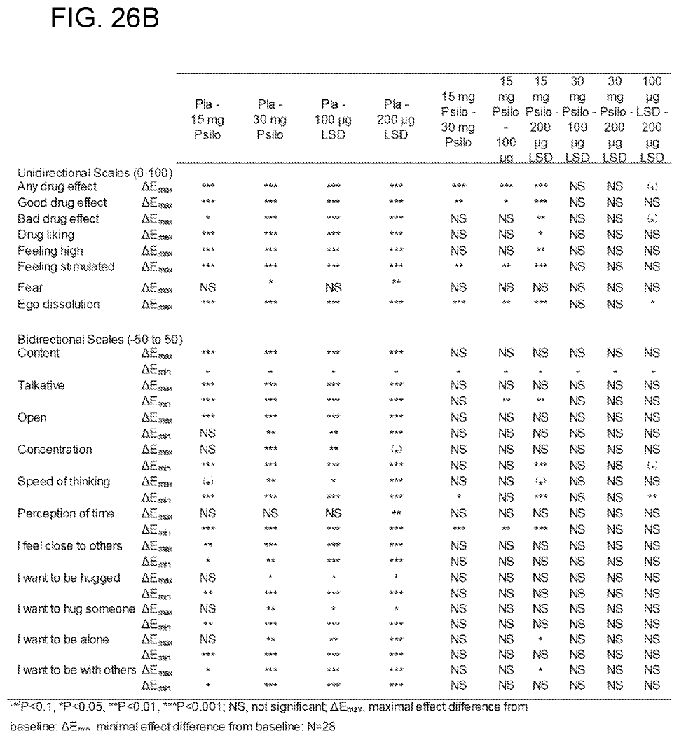

Subjective drug effects: Subjective effects over time on the VAS are shown in FIGS. 3-13. Maximal Values are shown in FIG. 26. Generally, the LSD doses of 100 and 200 μg and the psilocybin dose of 30 mg produced comparable subjective effects. Specifically, LSD 200 μg produced comparable positive drug effects to LSD 100 μg but the higher dose produced greater ego-dissolution (p=0.014) and near-significantly greater anxiety (p=0.054) consistent with a previous dose-response study on LSD in healthy subjects (Holze et al., 2021). The high 30 mg psilocybin dose produced comparable maximal subjective effects as the 100 or 200 μg LSD dose with no statistical differences on any of the VAS used. Nominally, maximal scores and effect-time curves after the 30 mg psilocybin dose were typically between those of the 100 and 200 μg LSD doses indicating that the 30 mg psilocybin dose would likely correspond to 150 μg of LSD although this was not directly tested. The 30 mg psilocybin dose produced significantly greater peak responses than the 15 mg psilocybin dose on the VAS any drug effect, good drug effect, stimulated, speed of thinking, perception of time, and ego dissolution. Effects of the 15 mg psilocybin dose were also significantly lower than those of the 200 μg LSD dose on the VAS any drug effects, good drug effect, stimulated, talkative, perception of time, and ego-dissolution. Thus, the psilocybin 15 mg dose produced clearly lower effects than the psilocybin 30 mg and both LSD doses. This also indicates that the dose-response curve is steeper in the 15-30 mg psilocybin dose range compared to the 100-200 μg LSD dose range where a plateau effect is already reached.

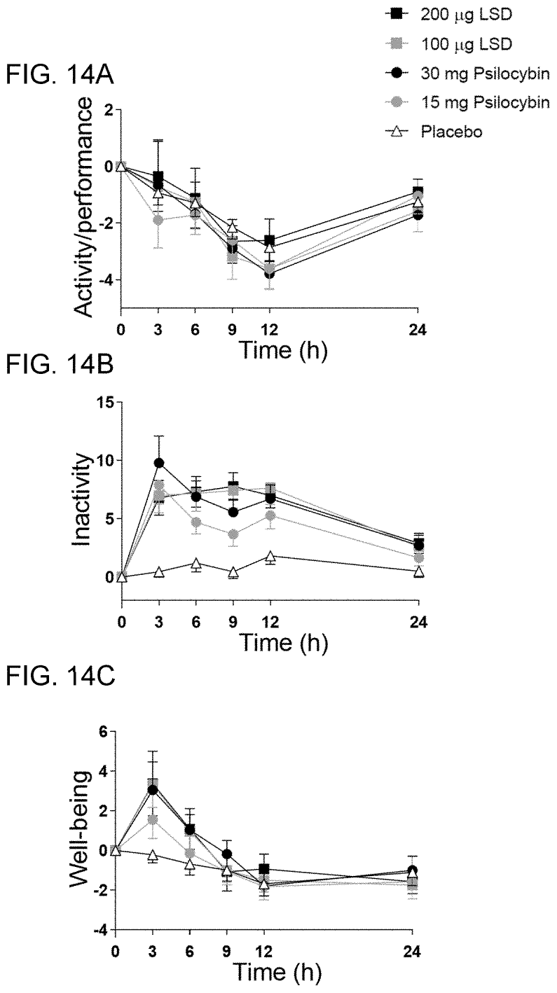

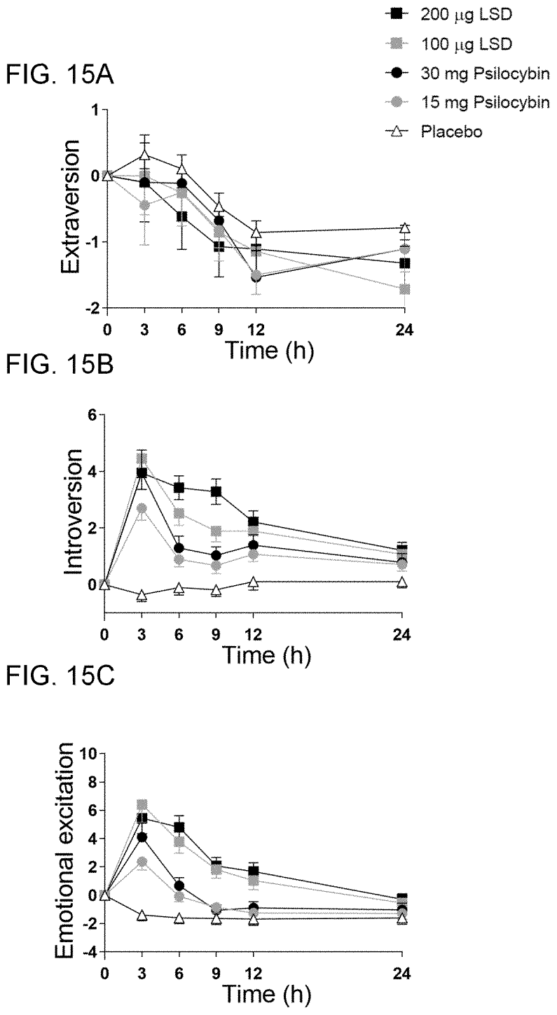

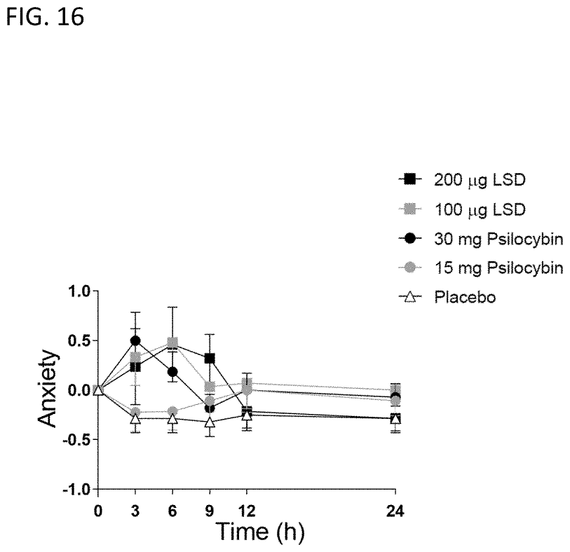

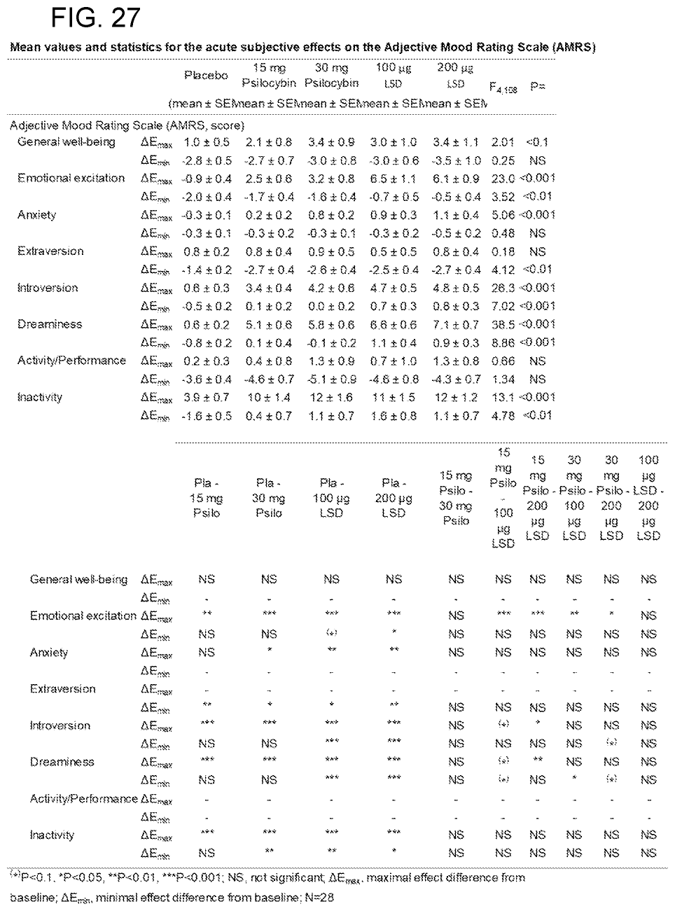

Effects on the AMRS are shown in FIGS. 14-16 and FIG. 27. All conditions nominally reduced activity ratings on the AMRS with no difference between placebo and either active drug condition (FIG. 14A). Both LSD and psilocybin significantly and similarly increased inactivation ratings compared with placebo and at all doses (FIG. 14B). None of the substances significantly altered well-being ratings although there were nominal increases compared with placebo (FIG. 14C). Both LSD and psilocybin increased introversion (FIG. 15B) and decrease extraversion (FIG. 15A) ratings at all doses and compared with placebo. Effects of LSD at 100 and 200 μg were greater than those of 15 mg or 30 mg of psilocybin indicating that LSD enhanced emotional excitation more than psilocybin at dose otherwise largely equivalent in terms of other subjective effects (FIG. 15C, FIG. 27). Both doses of LSD and the higher 30 mg psilocybin dose slightly but significantly increased self-rated anxiety in the AMRS (FIG. 16, FIG. 27).

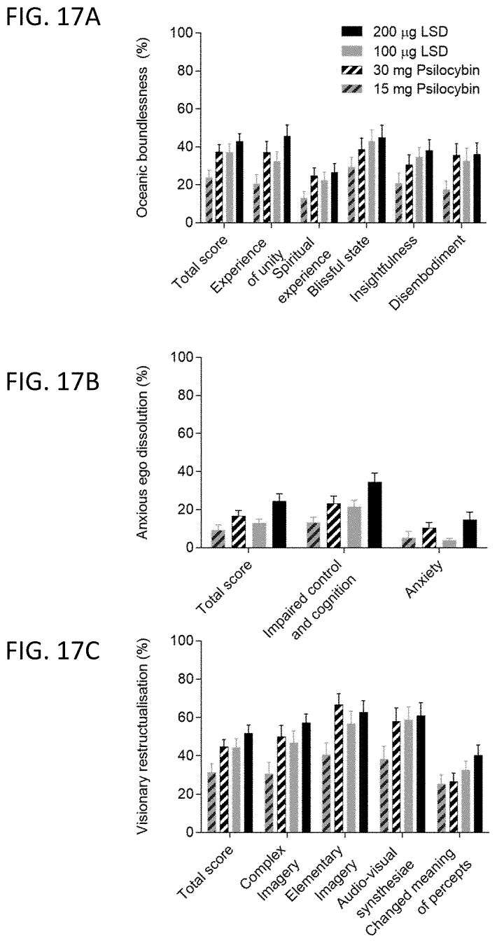

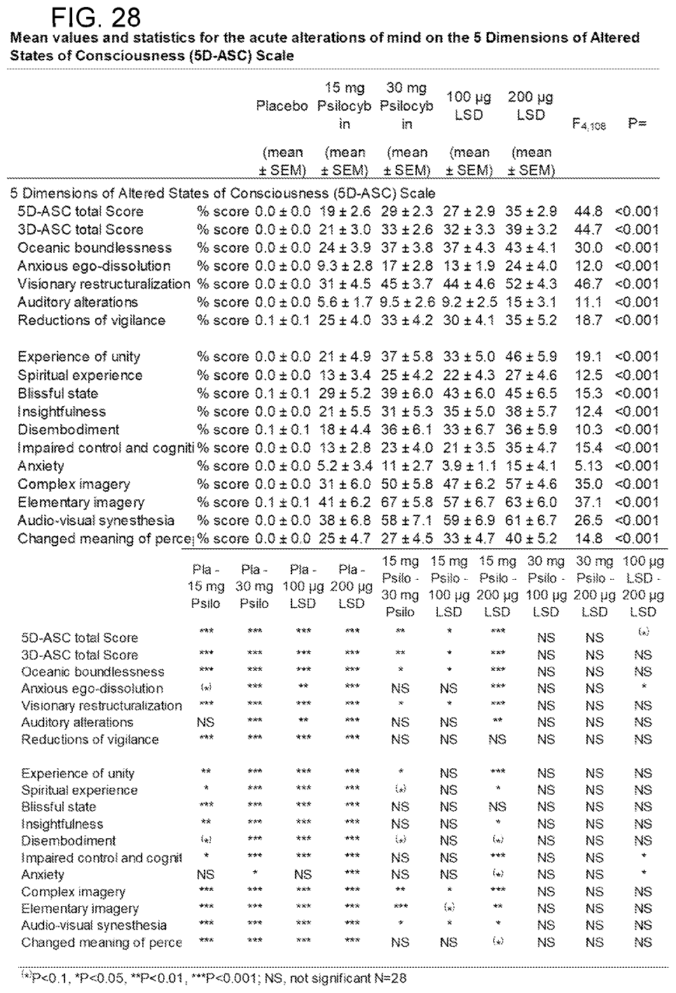

Effects on the 5D-ASC are shown in FIGS. 17A-17C and FIG. 28. Both LSD and psilocybin produced marked alterations of the mind in the 5D-ASC scale (FIGS. 17A-17C). LSD produced similar positive effects on the OB scale at both doses while the 200 μg dose of LSD produced greater AED (p=0.02) including greater impairments of control and cognition (p=0.04) and anxiety (p=0.03) than the 100 μg dose of LSD (FIGS. 17A-17C) consistent with a previous study on the effects of LSD (Holze et al., 2021). Psilocybin at 30 mg produced alterations of the mind that were nominally similar to those of LSD 100 μg and statistically not different from those of either LSD 100 μg or LSD 200 μg (FIGS. 17A-17C). Effects of the lower 15 mg psilocybin dose on the 5D-ASC were clearly lower than those of LSD at 100 μg of 200 μg or of the higher 30 mg psilocybin dose on most subscales (FIGS. 17A-17C).

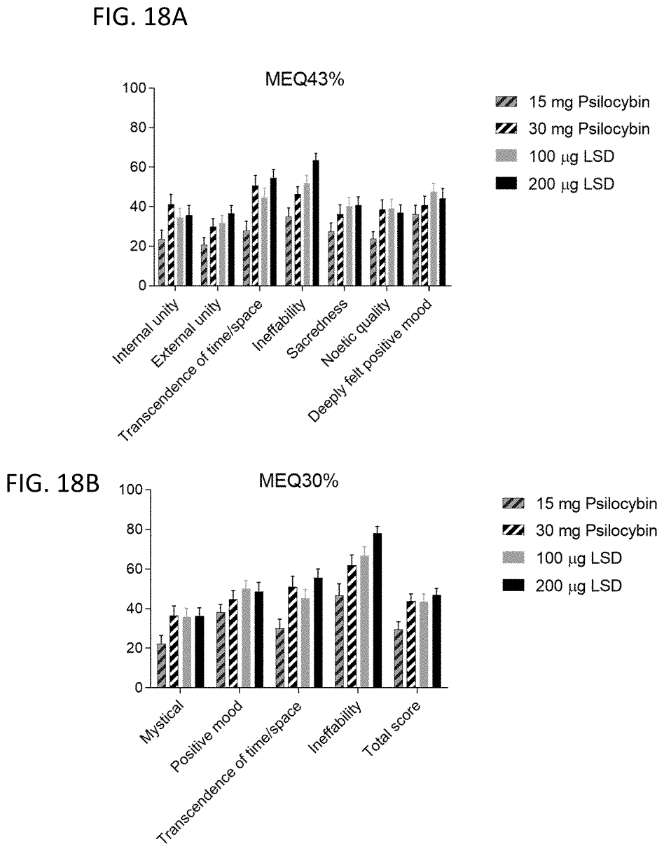

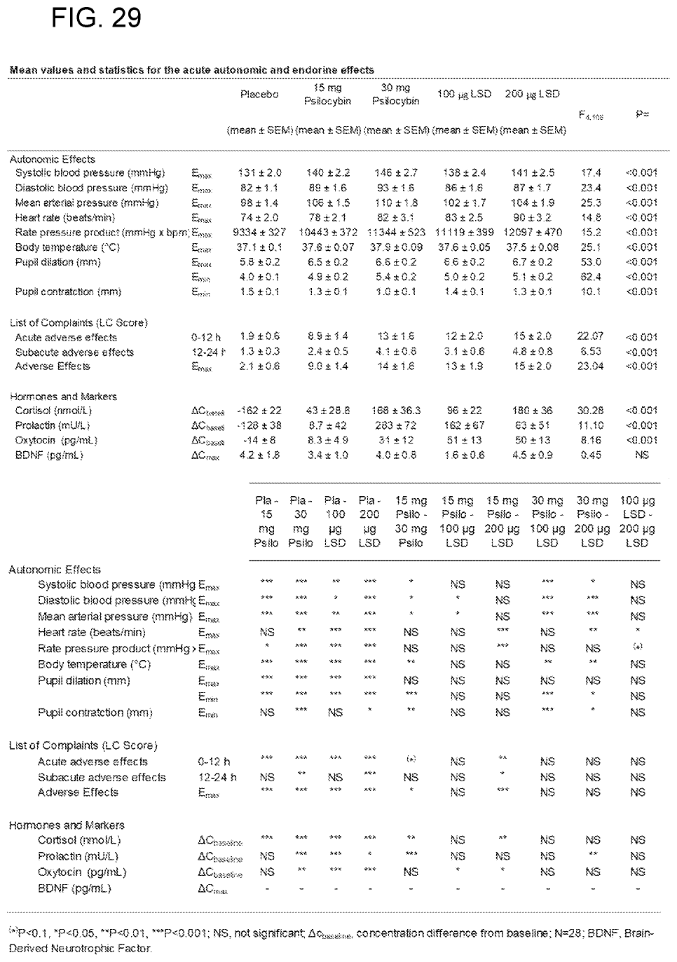

Effects on the MEQ are shown in FIGS. 17A-17C and FIG. 29. Generally, LSD and psilocybin produced similar and significant mystical experiences compared with placebo. The high psilocybin dose of 30 mg produces nominally similar increase than the 100 μg LSD dose and slightly lower effect than the 200 μg LSD dose. The high LSD 200 μg dose produced significantly greater ineffability ratings in the MEQ43 and MEQ30 (p=0.002 and p=0.015, respectively, FIGS. 18A-18B). Mystical experience ratings induced by psilocybin at the 15 mg dose were significantly smaller than those of either LSD dose or of 30 mg of psilocybin (FIGS. 18A-18B and FIG. 29).

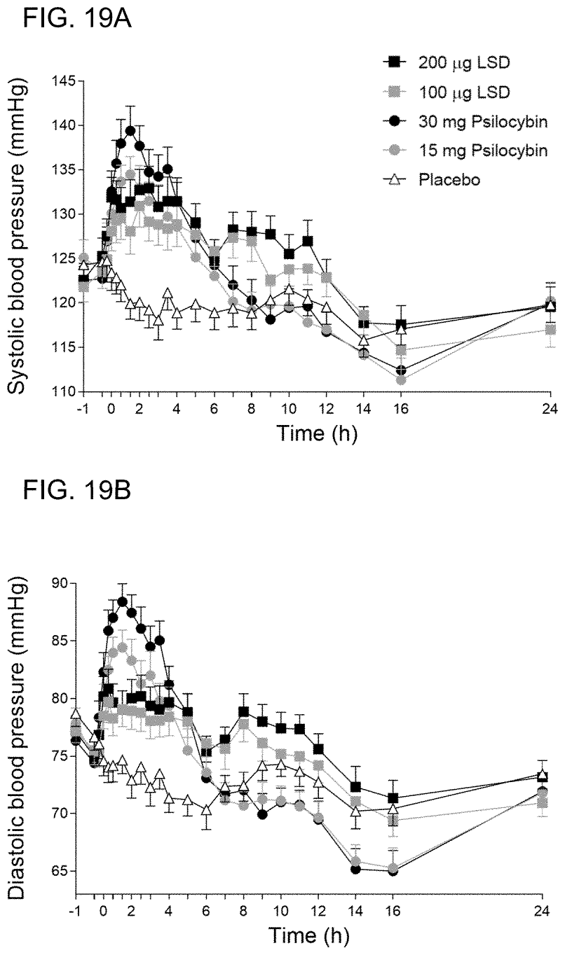

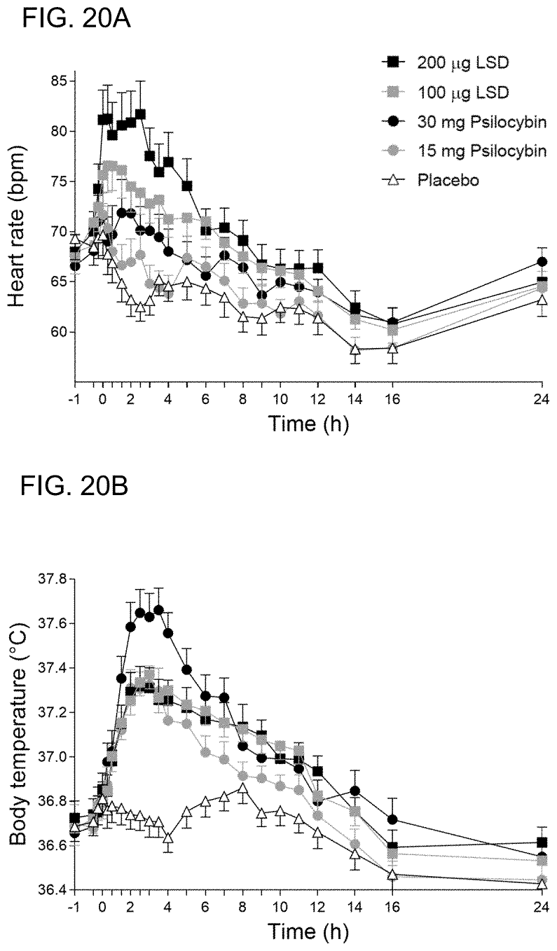

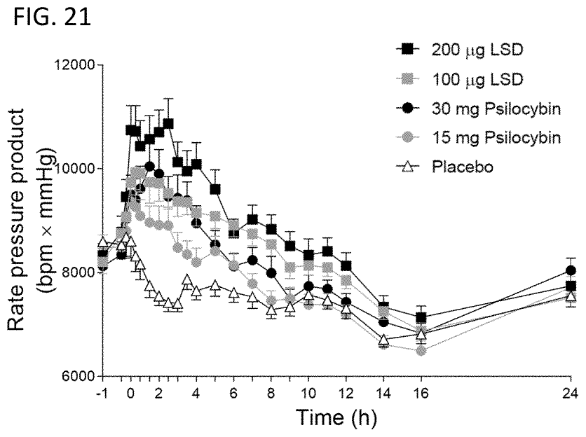

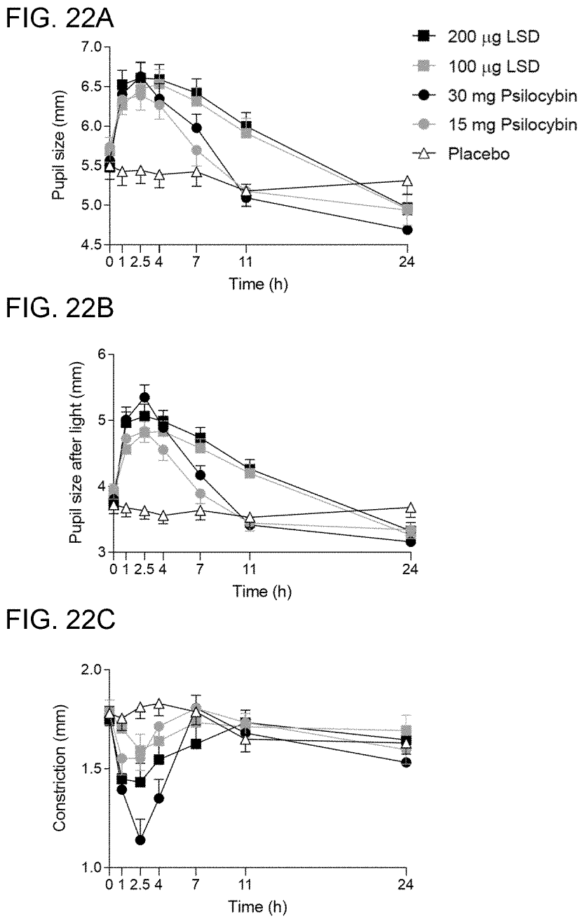

Autonomic effects are illustrated in FIG. 19-22 and FIG. 30. All substances produced significant increases in blood pressure, heart rate, rate-pressure product, body temperature and pupil size compared with placebo. Psilocybin produced greater increases in blood pressure and LSD produced greater increases in heart rate compared with the other substance. Specifically, psilocybin 30 mg increased systolic and diastolic blood pressure more than LSD 200 μg (p=0.04 and p<0.001, FIG. 19A and 19B, respectively). Vice versa, LSD 200 μg increased heart rate significantly more than psilocybin 30 mg (p=0.002, FIG. 20A). Both substances at the high dose similarly increased the rate pressure product, indicating comparable overall cardiovascular stimulation (FIG. 21). Both substances similarly increased pupil size (FIG. 22A) at all doses and reduced the reaction to light (FIG. 22B and 22C). The reduction of the pupillary constriction in response to light was more pronounced at the 30 mg psilocybin dose compared with psilocybin 15 mg (p=0.002), LSD 100 μg (p<0.001) and LSD 200 μg (p=0.02) (FIG. 22C).

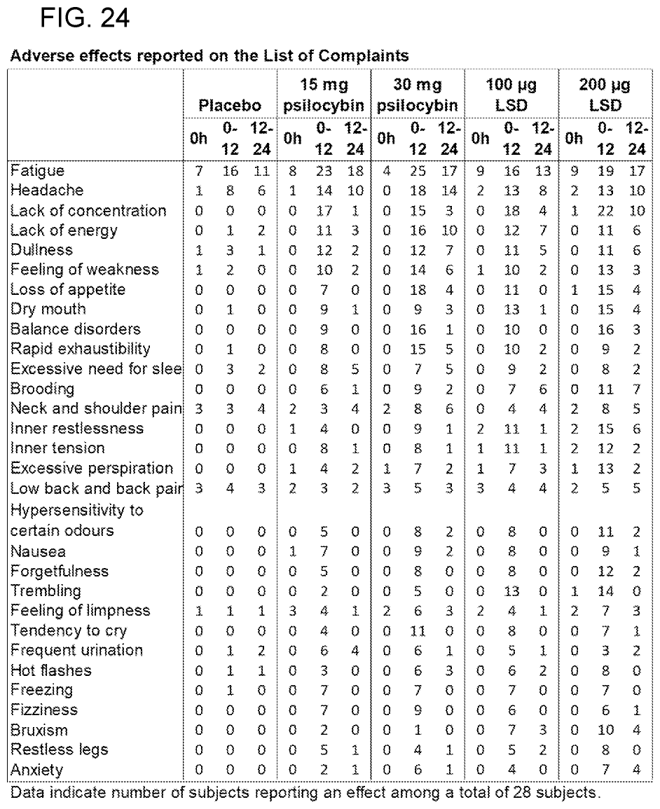

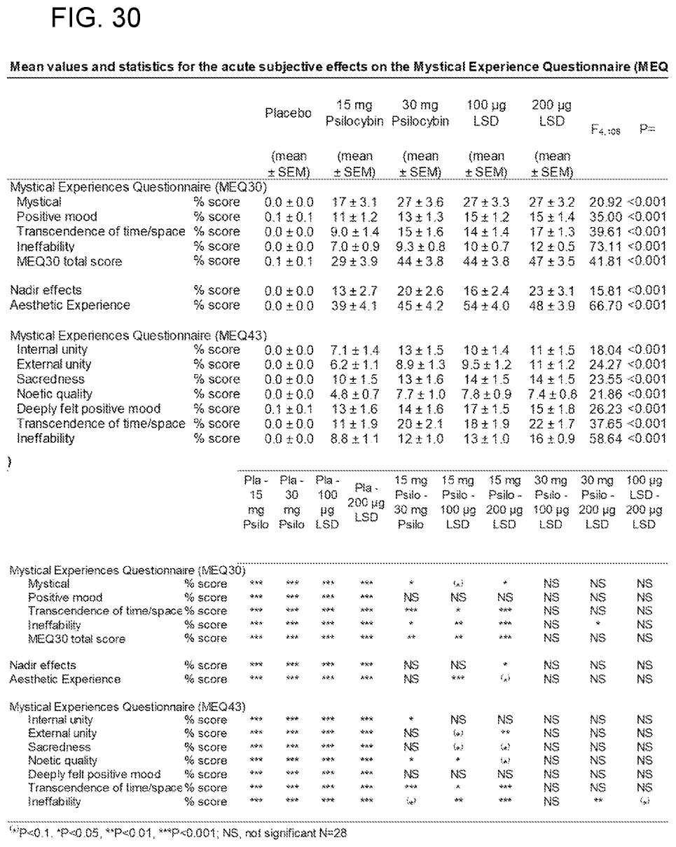

Adverse effects as systematically assessed by the list of complaints are shown in FIG. 24. The most frequent acute adverse effects of both LSD and psilocybin included fatigue, headache, lack of concentration, lack of energy, dullness, feeling week, loss of appetite, dry mouth, and impaired balance. Specifically, headache was reported by 18 and 13 subjects during (0-12 hours) the high dose psilocybin and LSD experience, respectively, but also in 8 cases after placebo. Dry mouth was reported by 9 and 15 subjects after psilocybin and LSD, respectively. Balance was disturbed in 16 subjects after psilocybin or LSD. See the Table in FIG. 24 for the most frequently reported complaints after each substance and dose. Overall, acute adverse effects were equally frequent after LSD and psilocybin (FIG. 30). The two LSD doses did not differ regarding total number of acute complaints (FIG. 30). Psilocybin 15 mg produced fewer acute adverse effects than LSD 100 μg (p<0.01) and there was also a trend toward fewer acute adverse effects than psilocybin 30 mg (p<0.1).

In addition to the adverse effects systematically assessed with the list of complaints, there were spontaneously reported adverse events after LSD or psilocybin administration as assessed in the evening of the treatment day or within 48 hours after treatment. These adverse after effects after psilocybin or LSD administration included headaches (four subjects after psilocybin and three after LSD), migraine attack (one subject after LSD), nosebleeds (one subject after psilocybin), low mood (two subjects after psilocybin and two subjects after LSD), nausea (two subjects after psilocybin and one subject after LSD), nightmares (one subject after psilocybin), restlessness (one subject after psilocybin and one subject after LSD), vivid dreams (one subject after LSD), insomnia (one subject after psilocybin), involuntary movement in lower extremities (one subject after LSD). Nine flash back episodes occurred in five subjects (one to 20 times within 72 hours after substance administration), five episodes occurred after LSD administration, four episodes occurred after psilocybin administration. Taken together, the type and amount of adverse events following acute psilocybin and LSD administration was comparable.

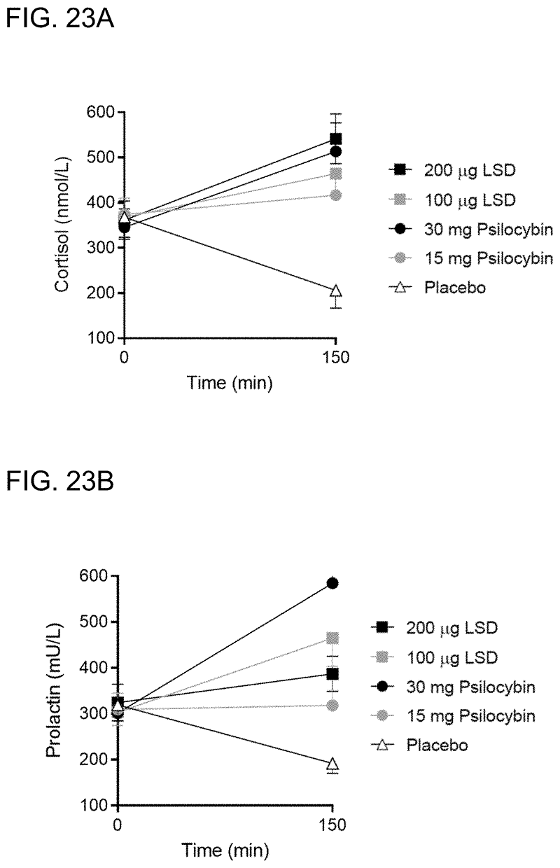

FIG. 23 shows effects of LSD and psilocybin on cortisol and prolactin (PRL). Both LSD and psilocybin significantly increased cortisol and PRL plasma concentrations (FIG. 23 and FIG. 30). Both PRL and cortisol increase indicate an increase in serotonergic activity (Seifritz et al., 1996) and the findings are consistent with the known mainly serotonergic effects of both LSD and psilocybin.

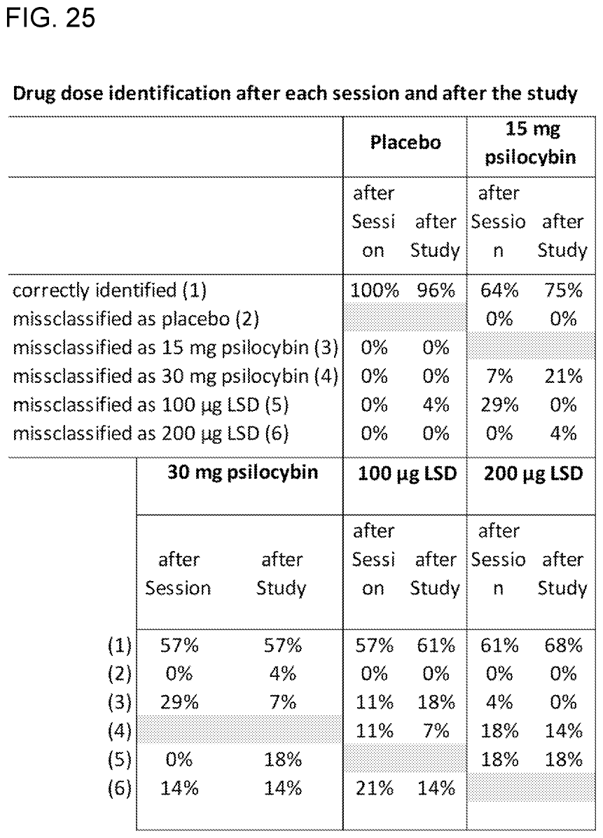

FIG. 25 shows the drug and dose blinding characteristics. Placebo could be distinguished well from active substance and was correctly identified in 96% of the sessions. 15 mg psilocybin was correctly identified in 64% after the session and mistaken for 30 mg psilocybin in 21% of the sessions. The 30 mg psilocybin dose was correctly identified in 57% of the sessions and if not mostly mistaken for 100 μg LSD. The 100 μg LSD session was correctly identified in 57% and if not mostly mistaken as 15 mg psilocybin (in 18%). The 200 μg LSD dose was correctly identified in 61% of the cases and mostly mistaken as 100 μg LSD (18%). Unblinding could potentially bias the study results due to expectations specifically linked to one or another substance or dose of substance. Overall, the relevant blinding between the different doses and substances was good and there was no significant unblinding for any substance or dose against another. This supports the validity of the study design used.

The present study example also determined the pharmacokinetics of LSD and psilocybin. However, this data will be included later and not critical for the present invention.

Similarly, effects of LSD and psilocybin on circulating BDNF concentrations were measured. BDNF is considered a possible marker of the therapeutic effects of LSD and other psychedelics as detailed previously (U.S. patent application Ser. No. 17/225,715, filed Apr. 8, 2021, titled: LSD dose identification). Thus, the concept of using BDNF as marker of outcome is not new. However, the specific levels obtained with a given dose of LSD and psilocybin and their comparability are further determined within the present invention. Data will be included later.

Taken together the present invention provides the basis for the dose equivalence proposed in FIG. 31. The LSD 200 μg dose was overall stronger than the 30 mg psilocybin dose indicating a dose equivalence to 40 mg of psilocybin. Vice versa a dose of 30 mg of psilocybin would correspond to 150 μg of LSD because the effects were largely between those of the 100 and the 200 μg of LSD. In this extrapolation, data from a more comprehensive response evaluation of LSD shown in (Holze et al., 2021) were also included. The commonly used dose of 25 mg of psilocybin likely corresponds to 125 μg of LSD base. These assumptions can be confirmed once the present invention is further developed and also including additional doses.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Barrett F S, Johnson M W, & Griffiths R R (2015). Validation of the revised Mystical Experience Questionnaire in experimental sessions with psilocybin. J Psychopharmacol 29: 1182-1190.

2. Barrett F S, Preller K H, Herdener M, Janata P, & Vollenweider F X (2017). Serotonin 2A Receptor Signaling Underlies LSD-induced Alteration of the Neural Response to Dynamic Changes in Music. Cereb Cortex: 1-12.

3. Barrett F S, Preller K H, Herdener M, Janata P, & Vollenweider F X (2018). Serotonin 2A Receptor Signaling Underlies LSD-induced Alteration of the Neural Response to Dynamic Changes in Music. Cereb Cortex 28: 3939-3950.

4. Bogenschutz M P (2013). Studying the effects of classic hallucinogens in the treatment of alcoholism: rationale, methodology, and current research with psilocybin. Curr Drug Abuse Rev 6:17-29.

5. Bogenschutz M P, Forcehimes A A, Pommy J A, Wilcox C E, Barbosa P C, & Strassman R J (2015). Psilocybin-assisted treatment for alcohol dependence: a proof-of-concept study. J Psychopharmacol 29: 289-299.

6. Carhart-Harris R L, Bolstridge M, Rucker J, Day C M, Erritzoe D, Kaelen M, Bloomfield M, Rickard J A, Forbes B, Feilding A, Taylor D, Pilling S, Curran V H, & Nutt D J (2016a). Psilocybin with psychological support for treatment-resistant depression: an open-label feasibility study. Lancet Psychiatry 3: 619-627.

7. Carhart-Harris R L, Kaelen M, Bolstridge M, Williams T M, Williams L T, Underwood R, Feilding A, & Nutt D J (2016b). The paradoxical psychological effects of lysergic acid diethylamide (LSD). Psychol Med 46: 1379-1390.

8. Carhart-Harris R L, Kaelen M, Whalley M G, Bolstridge M, Feilding A, & Nutt D J (2015). LSD enhances suggestibility in healthy volunteers. Psychopharmacology (Berl) 232: 785-794.

9. Carhart-Harris R L, Muthukumaraswamy S, Roseman L, Kaelen M, Droog W, Murphy K, Tagliazucchi E, Schenberg E E, Nest T, Orban C, Leech R, Williams L T, Williams T M, Bolstridge M, Sessa B, McGonigle J, Sereno M I, Nichols D, Hellyer P J, Hobden P, Evans J, Singh K D, Wise R G, Curran H V, Feilding A, & Nutt D J (2016c). Neural correlates of the LSD experience revealed by multimodal neuroimaging. Proc Natl Acad Sci USA 113: 4853-4858.

10. Davis A K, Barrett F S, May D G, Cosimano M P, Sepeda N D, Johnson M W, Finan P H, & Griffiths R R (2021). Effects of psilocybin-assisted therapy on major depressive disorder: a randomized clinical trial. JAMA Psychiatry 78: 481-489.

11. de Almeida R N, Galvao A C M, da Silva F S, Silva E, Palhano-Fontes F, Maia-de-Oliveira J P, de Araujo L B, Lobao-Soares B, & Galvao-Coelho N L (2019). Modulation of serum brain-derived neurotrophic factor by a single dose of ayahuasca: observation from a randomized controlled trial. Front Psychol 10: 1234.

12. Dittrich A (1998). The standardized psychometric assessment of altered states of consciousness (ASCs) in humans. Pharmacopsychiatry 31 (Suppl 2): 80-84.

13. Dolder P C, Schmid Y, Mueller F, Borgwardt S, & Liechti E (2016). LSD acutely impairs fear recognition and enhances emotional empathy and sociality. Neuropsychopharmacology 41: 2638-2646.

14. Dolder P C, Schmid Y, Steuer A E, Kraemer T, Rentsch K M, Hammann F, & Liechti M E (2017). Pharmacokinetics and pharmacodynamics of lysergic acid diethylamide in healthy subjects. Clin Pharmacokinetics 56: 1219-1230.

15. Garcia-Romeu A, Davis A K, Erowid F, Erowid E, Griffiths R R, & Johnson M W (2019). Cessation and reduction in alcohol consumption and misuse after psychedelic use. J Psychopharmacol: 269881119845793.

16. Garcia-Romeu A, Griffiths R R, & Johnson M W (2015). Psilocybin-occasioned mystical experiences in the treatment of tobacco addiction. Curr Drug Abuse Rev 7: 157-164.

17. Gasser P, Holstein D, Michel Y, Doblin R, Yazar-Klosinski B, Passie T, & Brenneisen R (2014). Safety and efficacy of lysergic acid diethylamide-assisted psychotherapy for anxiety associated with life-threatening diseases. J Nery Ment Dis 202: 513-520.

18. Gasser P, Kirchner K, & Passie T (2015). LSD-assisted psychotherapy for anxiety associated with a life-threatening disease: a qualitative study of acute and sustained subjective effects. J Psychopharmacol 29: 57-68.

19. Glennon R A, Raghupathi R, Bartyzel P, Teitler M, & Leonhardt S (1992). Binding of phenylalkylamine derivatives at 5-HT1C and 5-HT2 serotonin receptors: evidence for a lack of selectivity. J Med Chem 35: 734-740.

20. Griffiths R, Richards W, Johnson M, McCann U, & Jesse R (2008). Mystical-type experiences occasioned by psilocybin mediate the attribution of personal meaning and spiritual significance 14 months later. J Psychopharmacol 22: 621-632.

21. Griffiths R R, Johnson M W, Carducci M A, Umbricht A, Richards W A, Richards B D, Cosimano M P, & Klinedinst M A (2016). Psilocybin produces substantial and sustained decreases in depression and anxiety in patients with life-threatening cancer: a randomized double-blind trial. J Psychopharmacol 30: 1181-1197.

22. Griffiths R R, Richards W A, McCann U, & Jesse R (2006). Psilocybin can occasion mystical-type experiences having substantial and sustained personal meaning and spiritual significance. Psychopharmacology (Berl) 187: 268-283; discussion 284-292.

23. Grob C S, Danforth A L, Chopra G S, Hagerty M, McKay C R, Halberstadt A L, & Greer G R (2011). Pilot study of psilocybin treatment for anxiety in patients with advanced-stage cancer. Arch Gen Psychiatry 68: 71-78.

24. Haile C N, Murrough J W, Iosifescu D V, Chang L C, Al Jurdi R K, Foulkes A, Iqbal S, Mahoney J J, 3rd, De La Garza R, 2nd, Charney D S, Newton T F, & Mathew S J (2014). Plasma brain derived neurotrophic factor (BDNF) and response to ketamine in treatment-resistant depression. Int J Neuropsychopharmacol 17: 331-336.

25. Holze F, Duthaler U, Vizeli P, Muller F, Borgwardt S, & Liechti M E (2019). Pharmacokinetics and subjective effects of a novel oral LSD formulation in healthy subjects. Br J Clin Pharmacol 85: 1474-1483.

26. Holze F, Vizeli P, Ley L, Muller F, Dolder P, Stocker M, Duthaler U, Varghese N, Eckert A, Borgwardt S, & Liechti M E (2021). Acute dose-dependent effects of lysergic acid diethylamide in a double-blind placebo-controlled study in healthy subjects. Neuropsychopharmacology 46: 537-544.

27. Holze F, Vizeli P, Muller F, Ley L, Duerig R, Varghese N, Eckert A, Borgwardt S, & Liechti M E (2020). Distinct acute effects of LSD, MDMA, and D-amphetamine in healthy subjects. Neuropsychopharmacology 45: 462-471.

28. Holze F, Ley L, Müller F, Becker A M, Straumann I, Vizeli P, Kuehne S S, Roder M A, Duthaler U, Kolaczynksa K E, Varghese N, Eckert A, Liechti M E (2022). Direct comparison of the acute effects of lysergic acid diethylamide and psilocybin in a double-blind placebo-controlled study in healthy subjects. Neuropsychopharmacoly doi: 10.1038/s41386-022-01297-2.

29. Hysek C M, Vollenweider F X, & Liechti M E (2010). Effects of a b-blocker on the cardiovascular response to MDMA (ecstasy). Emerg Med J 27: 586-589.

30. Janke W, & Debus G (1978) Die Eigenschaftsworterliste. Hogrefe: Gottingen.

31. Johnson M W, Garcia-Romeu A, Cosimano M P, & Griffiths R R (2014). Pilot study of the 5-HT2AR agonist psilocybin in the treatment of tobacco addiction. J Psychopharmacol 28: 983-992.

32. Johnson M W, Garcia-Romeu A, & Griffiths R R (2016). Long-term follow-up of psilocybin-facilitated smoking cessation. Am J Drug Alcohol Abuse 43: 55-60.

33. Kaelen M, Barrett F S, Roseman L, Lorenz R, Family N, Bolstridge M, Curran H V, Feilding A, Nutt D J, & Carhart-Harris R L (2015). LSD enhances the emotional response to music. Psychopharmacology (Berl) 232: 3607-3614.

34. Kolaczynska K E, Liechti M E, & Duthaler U (2021). Development and validation of an LC-MS/MS method for the bioanalysis of psilocybin's main metabolites, psilocin and 4-hydroxyindole-3-acetic acid, in human plasma. J Chromatogr B Analyt Technol Biomed Life Sci 1164: 122486.

35. Kraehenmann R, Pokorny D, Aicher H, Preller K H, Pokorny T, Bosch O G, Seifritz E, & Vollenweider F X (2017a). LSD Increases Primary Process Thinking via Serotonin 2A Receptor Activation. Front Pharmacol 8: 814.

36. Kraehenmann R, Pokorny D, Vollenweider L, Preller K H, Pokorny T, Seifritz E, & Vollenweider F X (2017b). Dreamlike effects of LSD on waking imagery in humans depend on serotonin 2A receptor activation. Psychopharmacology (Berl) 234: 2031-2046.

37. Krebs T S, & Johansen P O (2013). Over 30 million psychedelic users in the United States. F1000 Res 2: 98.

38. Liechti M E (2017). Modern clinical research on LSD. Neuropsychopharmacology 42: 2114-2127.

39. Liechti M E, Dolder P C, & Schmid Y (2017). Alterations in conciousness and mystical-type experiences after acute LSD in humans. Psychopharmacology 234: 1499-1510.

40. Liechti M E, & Holze F (2021). Dosing psychedelics and MDMA. Curr Topics Behav Neurosci in press.

41. Ly C, Greb A C, Cameron L P, Wong J M, Barragan E V, Wilson P C, Burbach K F, Soltanzadeh Zarandi S, Sood A, Paddy M R, Duim W C, Dennis M Y, McAllister A K, Ori-McKenney K M, Gray J A, & Olson D E (2018). Psychedelics promote structural and functional neural plasticity. Cell Rep 23: 3170-3182.

42. Mueller F, Dolder P C, Schmidt A, Liechti M E, & Borgwardt S (2018). Altered network hub connectivity after acute LSD administration. Neuroimage Clin 18: 694-701.

43. Mueller F, Lenz C, Dolder P C, Harder S, Schmid Y, Lang U E, Liechti M E, & Borgwardt S (2017a). Acute effects of LSD on amygdala activity during processing of fearful stimuli in healthy subjects. Transl Psychiatry 7: e1084.

44. Mueller F, Lenz C, Dolder P C, Lang U E, Schmidt A, Liechti M E, & Borgwardt S (2017b). Increased thalamic resting-state connectivity as a core driver of LSD-induced hallucinations. Acta Psychiatr Scand 136: 648-657.

45. Passie T, Halpern J H, Stichtenoth D O, Emrich H M, & Hintzen A (2008). The pharmacology of lysergic acid diethylamide: a review. CNS Neurosci Ther 14: 295-314.

46. Preller K H, Burt J B, Ji J L, Schleifer C H, Adkinson B D, Stampfli P, Seifritz E, Repovs G, Krystal J H, Murray J D, Vollenweider F X, & Anticevic A (2018). Changes in global and thalamic brain connectivity in LSD-induced altered states of consciousness are attributable to the 5-HT2A receptor. Elife 7: e35082.

47. Preller K H, Herdener M, Pokorny T, Planzer A, Kraehenmann R, Staemfli P, Liechti M E, Seifritz E, & Vollenweider F X The role of the serotonin 2A receptor in the fabric and modulation of personal meaning in lysergic acid diethylamide (LSD)-induced states.

48. Preller K H, Herdener M, Pokorny T, Planzer A, Kraehenmann R, Stampfli P, Liechti M E, Seifritz E, & Vollenweider F X (2017). The fabric of meaning and subjective effects in LSD-induced states depend on serotonin 2A receptor activation Curr Biol 27: 451-457.

49. Preller K H, Razi A, Zeidman P, Stampfli P, Friston K J, & Vollenweider F X (2019). Effective connectivity changes in LSD-induced altered states of consciousness in humans. Proc Natl Acad Sci USA 116: 2743-2748.

50. Rickli A, Moning O D, Hoener M C, & Liechti M E (2016). Receptor interaction profiles of novel psychoactive tryptamines compared with classic hallucinogens. Eur Neuropsychopharmacol 26: 1327-1337.

51. Roseman L, Nutt D J, & Carhart-Harris R L (2017). Quality of acute psychedelic experience predicts therapeutic efficacy of psilocybin for treatment-resistant depression. Front Pharmacol 8: 974.

52. Ross S, Bossis A, Guss J, Agin-Liebes G, Malone T, Cohen B, Mennenga S E, Belser A, Kalliontzi K, Babb J, Su Z, Corby P, & Schmidt B L (2016). Rapid and sustained symptom reduction following psilocybin treatment for anxiety and depression in patients with life-threatening cancer: a randomized controlled trial. J Psychopharmacol 30: 1165-1180.

53. Schmid Y, Enzler F, Gasser P, Grouzmann E, Preller K H, Vollenweider F X, Brenneisen R, Mueller F, Borgwardt S, & Liechti M E (2015). Acute effects of lysergic acid diethylamide in healthy subjects. Biol Psychiatry 78: 544-553.

54. Schmid Y, Gasser P, Oehen P, & Liechti M E (2021). Acute subjective effects in LSD- and MDMA-assisted psychotherapy. J Psychopharmacol 35: 362-374.

55. Schmid Y, & Liechti M E (2018). Long-lasting subjective effects of LSD in normal subjects. Psychopharmacology (Berl) 235: 535-545.

56. Schmidt A, Mueller F, Lenz C, Dolder P C, Schmid Y, Zanchi D, Liechti M E, & Borgwardt S (2017). Acute LSD effects on response inhibition neuronal networks. Psychol Med 48: 1464-1473.

57. Seifritz E, Baumann P, Muller M J, Annen O, Amey M, Hemmeter U, Hatzinger M, Chardon F, & Holsboer-Trachsler E (1996). Neuroendocrine effects of a 20-mg citalopram infusion in healthy males: a placebo-controlled evaluation of citalopram as 5-HT function probe. Neuropsychopharmacology 14: 253-263.

58. Steuer A E, Poetzsch M, Stock L, Eisenbeiss L, Schmid Y, Liechti M E, & Kraemer T (2017). Development and validation of an ultra-fast and sensitive microflow liquid chromatography-tandem mass spectrometry (MFLC-MS/MS) method for quantification of LSD and its metabolites in plasma and application to a controlled LSD administration study in humans. Drug Test Anal 9: 788-797.

59. Studerus E, Gamma A, & Vollenweider F X (2010). Psychometric evaluation of the altered states of consciousness rating scale (OAV). PLoS One 5: e12412.

60. Tagliazucchi E, Roseman L, Kaelen M, Orban C, Muthukumaraswamy S D, Murphy K, Laufs H, Leech R, McGonigle J, Crossley N, Bullmore E, Williams T, Bolstridge M, Feilding A, Nutt D J, & Carhart-Harris R (2016). Increased global functional connectivity correlates with LSD-induced ego dissolution. Curr Biol 26: 1043-1050.

61. Vollenweider F X, & Preller K H (2020). Psychedelic drugs: neurobiology and potential for treatment of psychiatric disorders. Nat Rev Neurosci 21: 611-624.

62. Vollenweider F X, Vollenweider-Scherpenhuyzen M F, Babler A, Vogel H, & Hell D (1998). Psilocybin induces schizophrenia-like psychosis in humans via a serotonin-2 agonist action. Neuroreport 9: 3897-3902.

63. Zerssen D V (1976) Die Beschwerden-Liste. Münchener Informationssystem. Psychis: München.

What is claimed is:

1. A method of treating a patient with a psychedelic, including the steps of:
administering either a dose of LSD to the patient that is equivalent to a known dose of psilocybin with desired acute and therapeutic effects, or administering a dose of psilocybin to the patient that is equivalent to a known dose of LSD with desired acute and therapeutic effects; and
treating the patient.

2. The method of claim 1, wherein the patient is being treated for a condition chosen from the group consisting of depression, anxiety, and addiction.

3. The method of claim 1, further including the step of maximizing positive acute effects of the psychedelic wherein the positive subjective acute effects are chosen from the group consisting of good drug effect, drug liking, well-being, oceanic boundlessness, experience of unity, spiritual experience, blissful state, insightfulness, mystical-type experience positively experienced psychedelic effects, aspects of ego-dissolution, and combinations thereof.

4. The method of claim 1, further including the step of minimizing negative acute effects chosen from the group consisting of bad drug effect, anxiety, fear, increased ratings of anxious ego-dissolution, or acute paranoia, states of panic, and combinations thereof.

5. The method of claim 1, wherein the dose of LSD is 1-200 μg.

6. The method of claim 1, wherein the dose of psilocybin is 1-30 mg.

7. A method of treating a patient with LSD, including the steps of:
administering a dose of LSD to the patient equivalent to those of psilocybin known to be associated with positive long-term therapeutic outcomes.

8. A method of determining a dose of a psychedelic or the dose-equivalence to another psychedelic to be administered to an individual, including the steps of:
administering a dose of a psychedelic to an individual;
determining positive acute effects and negative acute effects in the individual;
adjusting the dose to provide more positive acute effects than negative acute effects in the individual; and
equating the dose to an equivalent dose of a second psychedelic.

9. The method of claim 8, wherein the individual is healthy and further including the step of predicting doses for unhealthy individuals.

10. The method of claim 8, further including the step of determining long term dosing and dose schedules for the psychedelic.
